(19) United States
(12) Patent Application Publication
(10) Pub. No.: US 2022/0188901 A1
(43) Pub. Date: Jun. 16, 2022

(54) SYSTEMS AND METHODS FOR DETERMINING AND PRESENTING PURCHASE RECOMMENDATIONS BASED ON PERSONAL GENETIC PROFILES

(71) Applicant: Orig3n, Inc., Boston, MA (US)

(72) Inventors: Robin Y. Smith, Boston, MA (US); Marcie A. Glicksman, Boston, MA (US); Sunil Anant Gupta, Boston, MA (US); Edward Joseph Coffey, Boston, MA (US); Kate Blanchard, Boston, MA (US); Stephanie Lento, Boston, MA (US); Shadrack Cgar Frazier, Boston, MA (US)

(21) Appl. No.: 17/688,406

(22) Filed: Mar. 7, 2022

Related U.S. Application Data
(63) Continuation of application No. 15/846,646, filed on Dec. 19, 2017, now abandoned.
(60) Provisional application No. 62/589,673, filed on Nov. 22, 2017.
(60) Provisional application No. 62/463,477, filed on Feb. 24, 2017.
(60) Provisional application No. 62/458,933, filed on Feb. 14, 2017.
(60) Provisional application No. 62/451,641, filed on Jan. 27, 2017.

(51) Int. Cl. G06Q 30/06; G16H 20/60; G06Q 30/02; G16B 20/20

(52) U.S. Cl. CPC G06Q 30/0631; G06Q 30/0643; G16H 20/60; G16B 20/20; G06Q 30/06; G06Q 30/0269; G06Q 30/0251

(57) ABSTRACT

Presented herein are systems and methods for automatically identifying and recommending purchases (e.g., in-app purchases) to a user based on the user's personal genetic profile. In certain embodiments, offers for such purchases are conveniently presented in the same software application (e.g., smartphone app or other computing device application) in which a user securely accesses his or her personalized genetic profile test results. Also presented herein are systems and methods for computer application developers to customize apps for presentation of recommended purchases based on a user's personal genetic profile. In certain embodiments, the systems and methods described herein provide for issuing of genetically tailored notifications to one or more mobile health devices of an individual based on an assessment of the individual's genetic profile. Such notifications, for example, can assist an individual in their adherence to particular recommended regiments, such as workout regimens.

### CROSS REFERENCE TO RELATED APPLICATIONS

This application is a continuation of application Ser. No. 15/846,646, filed Dec. 19, 2017, which claims the benefit of U.S. Provisional Application No. 62/451,641, filed Jan. 27, 2017, U.S. Provisional Application No. 62/458,933, filed Feb. 14, 2017, U.S. Provisional Application No. 62/463,477, filed Feb. 24, 2017, and U.S. Provisional Application No. 62/589,673, filed Nov. 22, 2017, the contents of each of which are hereby incorporated by reference herein in their entirety.

### FIELD OF THE INVENTION

This invention related generally to systems and methods for facilitating purchase recommendations to users of personal genetic profiles.

### BACKGROUND OF THE INVENTION

Genomes hold valuable information that can be used to better understand human biological characteristics and traits. Much research is being conducted to establish relationships between the human genome and biological characteristics and traits, in particular. For example, single nucleotide polymorphisms (SNPs) are specific sites identified in particular genes that influence biological characteristics and traits depending on the particular polymorphism of an individual. Different polymorphisms of the nucleotides at a specific site influence the relevant characteristic or trait differently. Relationships between the variants of SNPs and their corresponding biological characteristics and traits have been established and many more possible relationships are currently undiscovered and under investigation.

Personalized genetic profiles, such as LifeProfile™ offered by Orig3n, Inc. of Boston, Mass., provide SNP-based assessments of various characteristics and traits using simple cheek swab samples, providing secure, user-friendly, smartphone accessible test results. Individuals provide a biological sample and receive an assessment of their genetic profile that is accessible for review on their smartphones. Individuals can learn how their genome impacts their personal health characteristics, fitness characteristics, and dietary characteristics.

Many individuals take vitamins, supplements, and other over-the-counter or prescription medications on a recurring basis in order to enhance their wellbeing. Often, supplements and medications are taken to relieve chronic conditions. For example, some individuals take glucosamine to deal with joint pain. Supplements may also be taken to boost performance or function. For example, some individuals take supplements when weightlifting, such as nitric oxide, in order to boost their improvements in physique and strength. Individuals frequently self-prescribe such supplements based on personal research or physician recommendation. Thus, the decisions of individuals are made largely on qualitative information about how an individual feels or how the patient's condition, as described, sounds to a physician. Similarly, individuals choose fitness programs, meal plans, and other health and fitness-related regimens based on such qualitative information.

There is a need for systems and methods to assist individuals in the selection of supplements and health and fitness programs.

### SUMMARY

Presented herein are systems and methods for automatically identifying and recommending purchases (e.g., in-app purchases) to a user based on the user's personal genetic profile. In certain embodiments, offers for such purchases are conveniently presented in the same software application (e.g., smartphone app or other computing device application) in which a user securely accesses his or her personalized genetic profile test results. Also presented herein are systems and methods for computer application developers to customize apps for presentation of recommended purchases based on a user's personal genetic profile.

In certain embodiments, purchase recommendations for supplements are identified based on genotyping data for individuals. Biological samples provided by individuals are used to generate genotyping data for a range of biological characteristics. Genotyping data may be stored as a personal genetic profile assessment and graphically rendered to an individual in an assessment graphical user interface (e.g., via a smartphone app). Individuals use assessment graphical user interfaces to learn about how their personal genome affects their biological traits (e.g., health-related phenotypes). Examples include (i) nutritional characteristics (e.g., the way in which an individual's body processes different foods and nutrients), (ii) skin health, (iii) physical fitness, and (iv) personal behavior tendencies (e.g., empathy, risk of addiction, and tolerance for stress and pain).

Based on an individual's particular biological traits, the individual may benefit from or wish to take one or more supplements. For example, an individual may learn about the influence of his/her genetics on his/her ability to process certain foods and consequently benefit from, and wish to take, several supplements to assist in processing those foods. In an assessment graphical user interface, one or more purchase recommendations for supplements or links to purchase recommendations are identified and presented to an individual based on the individual's genotyping data. Thus, in certain embodiments, individuals can easily view recommendations for supplements to purchase based on their phenotype, with the ability to directly purchase or redirected to purchase the supplements from a graphical user interface.

In certain embodiments, a back-end graphical user interface provides developers an interface to create data structures that comprise purchase recommendation data. Developers input data for a purchase recommendation and associate the data with personal genetic profile products. Associations between purchase recommendation data and personal genetic profile products facilitate population of an assessment graphical user interface with purchase recommendations for individuals.

In one aspect, the invention is directed to a method for automatically identifying, and providing for graphical rendering and presentation to a user via graphical user interface (GUI), a purchase recommendation based on an assessment of an individual's genetic profile, the method comprising: (a) receiving (and/or accessing), by a processor of a computing device, genotyping data (e.g., a personal genetic profile assessment) corresponding to a biological sample of a user (e.g., one or more genotyping measurements of one or more SNPs, each SNP associated with one or more genes); (b) automatically identifying, by the processor, one or more recommended purchases based on the genotyping data for the user (e.g., the personal genetic profile assessment); and (c) causing, by the processor, graphical rendering of, for presentation to the user (e.g., for presentation on a user's mobile computing device), one or more icons and/or alphanumeric strings corresponding to the recommended purchase(s) (e.g., presenting the recommended purchase as an in-app purchase option, e.g., in the same app as presentation of the genotyping data results).

In certain embodiments, the received genotyping data comprises results of one or more genotyping measurements of one or more SNPs; at least one of the one or more recommended purchases identified in step (b) is associated with at least one of the measured SNPs; method comprises causing, by the processor, graphical rendering of an assessment GUI view comprising a graphical representation of the at least one measured SNP with which the at least one recommended purchase is associated [e.g., the graphical representation comprises graphics and/or text that identify the at least one measured SNP (e.g., via a SNP reference) and/or a gene (e.g., via a gene identifier) with which it is associated, along with a particular variant of the SNP that the user has (e.g., via a measurement outcome) and/or a qualifier associated with the variant]; and step (c) comprises causing graphical rendering of an icon and/or alphanumeric string corresponding to the at least one recommended purchase within the assessment GUI view in a manner that visually associates the icon and/or alphanumeric string with the graphical representation of the results of the genotyping measurement of the at least one measured SNP.

In certain embodiments, the method comprises: at step (c), causing graphical rendering of at least one of the one or more icons and/or alphanumeric strings as a selectable button corresponding to a particular recommended purchase; and associating, by the processor, the selectable button with a link (e.g., a weblink) to a predefined site of a specific merchant for purchasing the particular recommended purchase, such that a user selection of the selectable button initiates their purchase of the particular recommended purchase from the specific merchant.

In certain embodiments, the method comprises: receiving, by the processor, an indication of a user selection of the selectable button corresponding to the particular recommended purchase; automatically retrieving, by the processor, from a payment database, payment information for the user (e.g., credit card information; e.g., online payment service account information); and providing, by the processor, the user payment information to the specific vendor (e.g., such that no user interaction beyond a single click of the selectable button is required to complete their purchase of the particular recommended purchase).

In certain embodiments, the one or more recommended purchases comprise one or more supplements (e.g., nutritional supplements).

In certain embodiments, the one or more recommended purchases comprise one or more members selected from the group consisting of a meal program, a fitness program, a brain wave feedback program, a behavioral program (e.g., a focus program or an ADHD assistance program), and an individualized therapy.

In certain embodiments, the one or more members are individualized programs and/or therapies based on the genotyping data.

In certain embodiments, the automatically identifying step comprises automatically identifying, by the processor, one or more recommended purchases based on a variant of a SNP in a genome of the user.

In certain embodiments, the genotyping data received in step (a) comprises, for each of one or more SNPs measured via a genotyping measurement, a user-specific variant object that identifies and/or classifies a particular variant of the measured SNP that the user has; and step (b) comprises: accessing a purchase recommendation database comprising a plurality of purchase recommendation objects, each representing a specific potential recommended purchase, wherein each purchase recommendation object is associated with one or more stored variant objects; matching one or more of the user-specific variant objects to one or more of the stored variant objects to determine a set of one or more potential recommended purchase(s), each potential recommended purchase of the set represented by a purchase recommendation object associated with at least one of the one or more matching stored variant objects; and identifying, from the determined set of potential recommended purchases, the one or more recommended purchases.

In certain embodiments, for each of the one or more SNPs measured via a genotyping measurement, the user-specific variant object that identifies and/or classifies the particular variant of the measured SNP that the user has is associated with (i) a SNP reference that identifies the measured SNP and/or a gene identifier that identifies a gene with which the measured SNP is associated, and (ii) a measurement outcome that identifies the particular variant of the measured SNP that the user has and/or a qualifier that classifies the particular variant of the measured SNP that the user has; each of the one or more the stored variant objects is associated with (i) a SNP reference that identifies a specific SNP having a specific variant that the stored variant object represents and/or a gene identifier that identifies a gene with which the specific SNP is associated, and (ii) a measurement outcome that identifies the specific variant of the specific SNP that the variant object represents and/or a qualifier that classifies the specific variant of the specific SNP that stored variant object represents; and the matching the one or more of the user-specific variant objects to the one or more of the stored variant objects comprises, for each matching pair comprising a user-specific variant object matched to a stored variant object: (A) matching at least one of (i) the SNP reference associated with the user-specific variant object of the matching pair to the SNP reference associated with the stored variant object of the matching pair, and (ii) the gene identifier associated with the user-specific variant object of the matching pair to the gene identifier associated with the stored variant object of the matching pair; and (B) matching at least one of (i) the measurement outcome associated with the user-specific variant object of the matching pair to the measurement outcome associated with the stored variant object of the matching pair, and (ii) the qualifier associated with the user-specific variant object of the matching pair to the qualifier associated with the stored variant object of the matching pair.

In certain embodiments, the one or more recommended purchases comprises a custom meal program comprising one or more recommended recipes for the user, wherein the method comprises: determining, by the processor, based on the genotyping data for the user, a dietary profile of the user that represents dietary guidelines and/or taste preferences for the user; and determining, by the processor, the custom meal plan based on the user dietary profile.

In certain embodiments, the dietary profile comprises a set of user-specific dietary tags that identify specific common diets [e.g., to which the user should conform (e.g., alphanumeric strings such as “vegetarian”, “vegan”, “pescatarian”, “low-cholesterol”, “dairy-free”, “lactose-free”, “gluten-free”, “paled”, “low-sugar”, and the like)] and/or allergens [e.g., that the user should avoid (e.g., alphanumeric strings such as “dairy”, “peanut”, “nut”, “gluten”, and the like)] having been determined, by the processor, as associated with the user based on their genotyping data; and the determining the custom meal plan comprises: accessing a meal database comprising a plurality of predefined meal programs, each comprising a predefined set of one or more recipes, wherein each meal program is associated with one or more program-specific dietary tags that identify specific common diets (e.g., to which the meal program conforms) and/or allergens (e.g., that are present in one or more recipes of the meal program; e.g., that are absent from all the recipes of the meal program); and matching the user-specific dietary tags of the dietary profile with the program-dietary tags of the meal programs within the meal database.

In certain embodiments, the dietary profile comprises a set of user-specific dietary tags that identify specific common diets (e.g., to which the user should conform) and/or allergens (e.g., that the user should avoid) determined as associated with the user based on their genotyping data; and the determining the custom meal plan comprises: accessing a meal database comprising a plurality of stored recipes, wherein each stored recipe is associated with one or more recipe-specific dietary tags that identify specific common diets (e.g., to which the stored recipe conforms) and/or allergens (e.g., that are present in the stored recipe; e.g., that are absent from the stored recipe); and matching the user-specific dietary tags of the dietary profile with the recipe-specific dietary tags of the stored recipes within the meal database to determine a subset of stored recipes; and selecting, from the subset of stored recipes, the one or more recommended recipes of the custom meal program.

In certain embodiments, the dietary profile comprises a set of user-specific dietary tags that identify specific common diets (e.g., to which the user should conform) and/or allergens (e.g., that the user should avoid) determined as associated with the user based on their genotyping data; and the determining the custom meal plan comprises: (A) accessing a meal database comprising a plurality of stored recipes, each stored recipe comprising an ingredient list identifying a plurality of ingredients used in the stored recipe; and (B) determining, for each of a subset of one or more recipes of the plurality of stored recipes, based on the ingredient list that the stored recipe comprises, that (i) the stored recipe conforms to one or more common diets identified by one or more of the user-specific dietary tags and/or (ii) the stored recipe does not comprise any allergens identified by one or more of the user specific dietary tags; and (C) responsive to the determining in step (B), selecting from the subset of stored recipes determined in step (B), the one or more recommended recipes of the custom meal plan.

In certain embodiments, the method comprises, causing, by the processor, graphical rendering of, for presentation to the user (e.g., for presentation on a user's mobile computing device), presentation(s) of the one or more recommended recipes of the custom meal plan (e.g., for each of the one or more recommended recipes, causing the graphical rendering of any of: (i) a title of the recipe, (ii) a picture of the dish produced by the recipe, (iii) a list of ingredients of the recipe, (iv) a cooking procedure of the recipe).

In certain embodiments, the custom meal plan comprises an identification of one or more specific restaurants and/or food delivery services through which the user can obtain at least one recipe of the recommended recipes (e.g., participating restaurants and/or participating food delivery services that provide recipe information for storage in the meal database).

In certain embodiments, the one or more recommended purchases comprises a custom fitness program comprising one or more recommended workout classes, each of which is associated with one or more specific variants and/or qualifiers of one or more specific SNPs.

In certain embodiments, the one or more recommended purchases comprises a custom fitness program for the user comprising one or more recommended workout classes, wherein the method comprises: determining, by the processor, based on the genotyping data for the user, a physical fitness profile of the user that represents particular types of physical exercises that the user should emphasize and/or avoid based on their unique fitness needs and/or predisposition to particular types of injury; and determining, by the processor, the one or more recommended workout classes based on the user physical fitness profile.

In certain embodiments, the physical fitness profile comprises a set of user-specific fitness tags that identify specific workout classifications (e.g., that are recommended for the user; e.g., that the user should avoid)(e.g., alphanumeric strings such as “HIIT”, “aerobic”; “cardio”; “high intensity”, “flexibility”) having been determined, by the processor, as associated with (e.g., beneficial to) the user based on their genotyping data; and the determining the one or more recommended workouts classes comprises: accessing a workout class database comprising a plurality of stored workout classes each associated with one or more program-specific fitness tags that identify specific classifications that the workout class falls under; and matching the user-specific fitness tags of the physical fitness profile with the program-specific fitness tags of the workout classes within the database.

In certain embodiments, the method comprises, causing, by the processor, graphical rendering of, for presentation to the user (e.g., for presentation on a user's mobile computing device), graphics and/or text representing additional information associated with the workout class (e.g., one or more times when the class is offered; e.g., one or more locations (e.g., of specific gyms) at which the class is offered; e.g., a cost of the class; e.g., a link to sign up for the class).

In certain embodiments, the method further comprises receiving (and/or accessing), by the processor, mobile health data recorded by a mobile health device of the user, and wherein the method comprises automatically identifying, by the processor, one or more recommended purchases based on the genotyping data for the user and the received mobile health data.

In certain embodiments, the one or more recommended purchases comprises one or more mobile health devices (and/or one or more software apps operating on a mobile health device).

In certain embodiments, the one or more recommended purchases comprises a first recommended purchase (e.g., a meal program, a fitness program, a brain wave feedback program, or a behavioral program) and at least one of one or more mobile health devices (and/or one or more software apps operating on a mobile health device) associated with the first recommended purchase (e.g. that facilitate use of the first recommended purchase by the user).

In another aspect, the invention is directed to a system for automatically identifying, and providing for graphical rendering and presentation to a user via graphical user interface (GUI), a purchase recommendation based on an assessment of an individual's genetic profile, the system comprising: a processor; and a memory having instructions stored thereon, wherein the instructions, when executed by the processor, cause the processor to: (a) receive (and/or access) genotyping data corresponding to a biological sample of a user (e.g., one or more genotyping measurements of one or more SNPs, each SNP associated with one or more genes); (b) automatically identify one or more recommended purchases based on the genotyping data for the user; and (c) cause graphical rendering of, for presentation to the user (e.g., for presentation on a user's mobile computing device), one or more icons and/or alphanumeric strings corresponding to the recommended purchase(s) (e.g., presenting the recommended purchase as an in-app purchase option, e.g., in the same app as presentation of the genotyping data results).

In certain embodiments, the received genotyping data comprises results of one or more genotyping measurements of one or more SNPs; at least one of the one or more recommended purchases identified in step (b) is associated with at least one of the measured SNPs; the instructions cause the processor to cause graphical rendering of an assessment GUI view comprising a graphical representation of the at least one measured SNP with which the at least one recommended purchase is associated [e.g., the graphical representation comprises graphics and/or text that identify the at least one measured SNP and/or a gene with which it is associated, along with a particular variant of the SNP that the user has and/or a qualifier associated with the variant]; and at step (c) the instructions cause the processor to cause graphical rendering of an icon and/or alphanumeric string corresponding to the at least one recommended purchase within the assessment GUI view in a manner that visually associates the icon and/or alphanumeric string with the graphical representation of the results of the genotyping measurement of the at least one measured SNP.

In certain embodiments, the instructions cause the processor to: at step (c), cause graphical rendering of at least one of the one or more icons and/or alphanumeric strings as a selectable button corresponding to a particular recommended purchase; and associate the selectable button with a link (e.g., a weblink) to a predefined site of a specific merchant for purchasing the particular recommended purchase, such that a user selection of the selectable button initiates their purchase of the particular recommended purchase from the specific merchant.

In certain embodiments, the instructions cause the processor to: receive an indication of a user selection of the selectable button corresponding to the particular recommended purchase; automatically retrieve from a payment database, payment information for the user (e.g., credit card information; e.g., online payment service account information); and provide the user payment information to the specific vendor (e.g., such that no user interaction beyond a single click of the selectable button is required to complete their purchase of the particular recommended purchase).

In certain embodiments, the one or more recommended purchases comprise one or more supplements (e.g., nutritional supplements).

In certain embodiments, the one or more recommended purchases comprise one or more members selected from the group consisting of a meal program, a fitness program, a brain wave feedback program, a behavioral program (e.g., a focus program, an ADHD assistance program), and an individualized therapy.

In certain embodiments, the one or more members are individualized programs and/or therapies based on the genotyping data.

In certain embodiments, the instructions, when executed by the processor, cause the processor to: automatically identify the one or more recommended purchases based on a variant of a SNP in a genome of the user.

In certain embodiments, the genotyping data received in step (a) comprises, for each of one or more SNPs measured via genotyping measurement, a user-specific variant object that identifies and/or classifies a particular variant of the measured SNP that the user has; and at step (b) the instructions cause the processor to: access a purchase recommendation database comprising a plurality of purchase recommendation objects, each representing a specific potential recommended purchase, wherein each purchase recommendation object is associated with one or more stored variant objects; match one or more of the user-specific variant objects to one or more of the stored variant objects to determine a set of one or more potential recommended purchase(s), each potential recommended purchase of the set represented by a purchase recommendation object associated with at least one of the one or more matching stored variant objects; and identify, from the determined set of potential recommended purchases, the one or more recommended purchases.

In certain embodiments, for each of the one or more SNPs measured via a genotyping measurement, the user-specific variant object that identifies and/or classifies the particular variant of the measured SNP that the user has is associated with (i) a SNP reference that identifies the measured SNP and/or a gene identifier that identifies a gene with which the measured SNP is associated, and (ii) a measurement outcome that identifies the particular variant of the measured SNP that the user has and/or a qualifier that classifies the particular variant of the measured SNP that the user has; each of the one or more the stored variant objects is associated with (i) a SNP reference that identifies a specific SNP having a specific variant that the stored variant object represents and/or a gene identifier that identifies a gene with which the specific SNP is associated, and (ii) a measurement outcome that identifies the specific variant of the specific SNP that the variant object represents and/or a qualifier that classifies the specific variant of the specific SNP that stored variant object represents; and the instructions cause the processor to match the one or more of the user-specific variant objects to the one or more of the stored variant objects by, for each matching pair comprising a user-specific variant object matched to a stored variant object: (A) matching at least one of (i) the SNP reference associated with the user-specific variant object of the matching pair to the SNP reference associated with the stored variant object of the matching pair, and (ii) the gene identifier associated with the user-specific variant object of the matching pair to the gene identifier associated with the stored variant object of the matching pair; and (B) matching at least one of (i) the measurement outcome associated with the user-specific variant object of the matching pair to the measurement outcome associated with the stored variant object of the matching pair, and (ii) the qualifier associated with the user-specific variant object of the matching pair to the qualifier associated with the stored variant object of the matching pair.

In certain embodiments, the one or more recommended purchases comprises a custom meal program comprising one or more recommended recipes for the user, wherein the instructions cause the processor to: determine, based on the genotyping data for the user, a dietary profile of the user that represents dietary guidelines and/or taste preferences for the user; determine the custom meal plan based on the user dietary profile.

In certain embodiments, the dietary profile comprises a set of user-specific dietary tags that identify specific common diets [e.g., to which the user should conform (e.g., alphanumeric strings such as “vegetarian”, “vegan”, “pescatarian”, “dairy-free”, “lactose-free”, “gluten-free”, “paleo”, “low-sugar”, and the like)] and/or allergens [e.g., that the user should avoid (e.g., alphanumeric strings such as “dairy”, “peanut”, “nut”, “gluten”, and the like)] having been determined, by the processor, as associated with the user based on their genotyping data; and the instructions cause the processor to determine the custom meal plan by: accessing a meal database comprising a plurality of predefined meal programs, each comprising a predefined set of one or more recipes, wherein each meal program is associated with one or more program-specific dietary tags that identify specific common diets (e.g., to which the meal program conforms) and/or allergens (e.g., that are present in one or more recipes of the meal program; e.g., that are absent from all the recipes of the meal program); and matching the user-specific dietary tags of the dietary profile with the program-dietary tags of the meal programs within the meal database.

In certain embodiments, the dietary profile comprises a set of user-specific dietary tags that identify specific common diets (e.g., to which the user should conform) and/or allergens (e.g., that the user should avoid) determined as associated with the user based on their genotyping data; and the instructions cause the processor to determine the custom meal plan by: accessing a meal database comprising a plurality of stored recipes, wherein each stored recipe is associated with one or more recipe-specific dietary tags that identify specific common diets (e.g., to which the stored recipe conforms) and/or allergens (e.g., that are present in the stored recipe; e.g., that are absent from the stored recipe); and matching the user-specific dietary tags of the dietary profile with the recipe-specific dietary tags of the stored recipes within the meal database to determine a subset of stored recipes; and selecting, from the subset of stored recipes, the one or more recommended recipes of the custom meal program.

In certain embodiments, the dietary profile comprises a set of user-specific dietary tags that identify specific common diets (e.g., to which the user should conform) and/or allergens (e.g., that the user should avoid) determined as associated with the user based on their genotyping data; and the instructions cause the processor to determine the custom meal plan by: (A) accessing a meal database comprising a plurality of stored recipes, each stored recipe comprising an ingredient list identifying a plurality of ingredients used in the stored recipe; and (B) determining, for each of a subset of one or more recipes of the plurality of stored recipes, based on the ingredient list that the stored recipe comprises, that (i) the stored recipe conforms to one or more common diets identified by one or more of the user-specific dietary tags and/or (ii) the stored recipe does not comprise any allergens identified by one or more of the user specific dietary tags; and (C) responsive to the determining in step (B), selecting from the subset of stored recipes determined in step (B), the one or more recommended recipes of the custom meal plan.

In certain embodiments, the instructions cause the processor to cause graphical rendering of, for presentation to the user (e.g., for presentation on a user's mobile computing device), representation(s) of the one or more recommended recipes of the custom meal plan (e.g., for each of the one or more recommended recipes, causing the graphical rendering of any of: (i) a title of the recipe, (ii) a picture of the dish produced by the recipe, (iii) a list of ingredients of the recipe, (iv) a cooking procedure of the recipe).

In certain embodiments, the custom meal plan comprises an identification of one or more specific restaurants and/or food delivery services through which the user can obtain at least one recipe of the recommended recipes (e.g., participating restaurants and/or participating food delivery services that provide recipe information for storage in the meal database).

In certain embodiments, the one or more recommended purchases comprises a custom fitness program comprising one or more recommended workout classes, each of which is associated with one or more specific variants and/or qualifiers of one or more specific SNPs.

In certain embodiments, the one or more recommended purchases comprises a custom fitness program for the user comprising one or more recommended workout classes, wherein the instructions cause the processor to: determine based on the genotyping data for the user, a physical fitness profile of the user that represents particular types of physical exercises that the user should emphasize and/or avoid based on their unique fitness needs and/or predisposition to particular types of injury; and determine the one or more recommended workout classes based on the user physical fitness profile.

In certain embodiments, the physical fitness profile comprises a set of user-specific fitness tags that identify specific workout classifications (e.g., that are recommended for the user; e.g., that the user should avoid)(e.g., alphanumeric strings such as “HIIT”, “aerobic”; “cardio”; “high intensity”, “flexibility”) having been determined, by the processor, as associated with the user based on their genotyping data; and the instructions cause the processor to determine the one or more recommended workouts classes by: accessing a workout class database comprising a plurality of stored workout classes each associated with one or more program-specific fitness tags that identify specific classifications that the workout class falls under; and matching the user-specific fitness tags of the physical fitness profile with the program-specific fitness tags of the workout classes within the database.

In certain embodiments, the instructions cause the processor to cause graphical rendering of, for presentation to the user (e.g., for presentation on a user's mobile computing device), representation(s) of additional information associated with the workout class (e.g., one or more times when the class is offered; e.g., one or more locations (e.g., of specific gyms) at which the class is offered; e.g., a cost of the class; e.g., a link to sign up for the class).

In certain embodiments, the instructions, when executed by the processor, cause the processor to: receive (and/or access) mobile health data recorded by a mobile health device of the user; and automatically identify the one or more recommended purchases based on the genotyping data for the user and the received the mobile health data.

In certain embodiments, the one or more recommended purchases comprises one or more mobile health devices (and/or one or more software apps operating on a mobile health device).

In certain embodiments, the one or more recommended purchases comprises a first recommended purchase (e.g., a meal program, a fitness program, a brain wave feedback program, or a behavioral program) and one or more mobile health devices (and/or one or more software apps operable on a mobile health device) associated with the first recommended purchase (e.g. that facilitate use of the first recommended purchase by the user).

In another aspect, the invention is directed to a method for creating purchase recommendation objects, the method comprising: (a) presenting, by a processor of a computing device, a graphical user interface element (e.g., widget) for creation of a purchase recommendation object that corresponds to a recommended purchase (e.g., a supplement, a program, and/or a mobile health device) recommended for use by individuals based on their genotyping data (e.g., for use by individuals with a particular variant of a gene), wherein the purchase recommendation object comprises one or more icons and/or alphanumeric strings that describe (e.g., identify) the recommended purchase; (b) receiving, by the processor, via the graphical user interface element, the purchase recommendation object; (c) receiving, by the processor, via the graphical user interface element, a developer selection of one or more stored genomic objects (e.g., gene objects, SNP objects, variant objects) that correspond to one or more genomic constituents (e.g., genes, SNPs, variants) for which the recommended purchase is recommended; (d) associating, by the processor, the purchase recommendation object with the one or more stored genomic objects; and (e) storing, by the processor, the purchase recommendation object and the association(s) between the purchase recommendation object and the one or more stored genomic objects for further updating and/or retrieval (e.g., in displaying the purchase recommendation object to an individual).

In certain embodiments, the purchase recommendation object comprises a link for purchasing the recommended purchase (e.g., in an app or on an external webpage).

In certain embodiments, the recommended purchase comprises one or more dietary supplements and/or nutritional supplements.

In certain embodiments, the recommended purchase comprises a member selected from the group consisting of a meal program, a fitness program, a brain wave feedback program, a behavioral program (e.g., a focus program, an ADHD assistance program), and an individualized therapy.

In certain embodiments, the recommended purchase is recommended for use by an individual based on his or her genotyping data and health data for the individual (e.g., health data trackable by a mobile health device).

In another aspect, the invention is directed to a system for linking purchase recommendations with personal genetic profile products, the system comprising: a processor; and a non-transitory computer readable medium having instructions stored thereon, wherein the instructions, when executed by the processor, cause the processor to: present a graphical user interface element (e.g., widget) for creation of a purchase recommendation object that corresponds to a recommended purchase, recommended for use by individuals with a particular variant of a gene, wherein the purchase recommendation object comprises one or more icons and/or alphanumeric strings that describe (e.g., identify) the recommended purchase; receive, via the graphical user interface element, the purchase recommendation object; receive, via the graphical user interface element, a developer selection of one or more stored genomic objects (e.g., gene objects, SNP objects, variant objects) that correspond to one or more genomic constituents (e.g., genes, SNPs, variants) for which the recommended purchase is recommended; associate the purchase recommendation object with the one or more stored genomic objects; and store the purchase recommendation object and the association(s) between the purchase recommendation object and the one or more stored genomic objects for further updating and/or retrieval (e.g., in displaying the purchase recommendation object to an individual).

In certain embodiments, the purchase recommendation object comprises a link for purchasing the recommended purchase (e.g., in an app or on an external webpage).

In certain embodiments, the recommended purchase comprises one or more dietary supplements and/or nutritional supplements.

In certain embodiments, the recommended purchase comprises a member selected from the group consisting of a meal program, a fitness program, a brain wave feedback program, a behavioral program (e.g., a focus program, an ADHD assistance program), and an individualized therapy.

In certain embodiments, the recommended purchase is recommended for use by an individual based on his or her genotyping data and health data for the individual (e.g., health data trackable by a mobile health device).

In another aspect, the invention is directed to a method for automatically providing genetically tailored notifications to one or more mobile health devices of an individual based on an assessment of the individual's genetic profile, the method comprising: receiving (and/or accessing), by a processor of a computing device, genotyping data (e.g., a personal genetic profile assessment) corresponding to a biological sample of a user (e.g., one or more genotyping measurements of one or more SNPs, each SNP associated with one or more genes); automatically determining, by the processor, a feedback recommendation (e.g. a recommendation to reduce physical activity level; e.g. a recommended physical activity level; e.g. a recommended number of meditation sessions) based on the genotyping data for the user (e.g. the personal genetic profile assessment); and causing, by the processor, creation of, for presentation to the user (e.g. for presentation on a user mobile health device; e.g. for presentation on a user computing device), a notification (e.g. a graphically rendered notification comprising one or more icons and/or alphanumeric strings displayed on a user mobile health device; e.g. an auditory notification (e.g. an alarm; e.g. an audio message); e.g. a haptic cue (e.g. a vibration), e.g. any combination of a graphically rendered notification, an auditory notification and a haptic cue) corresponding to the feedback recommendation.

In certain embodiments, the method comprises: receiving (and/or accessing) (e.g. via a network; e.g. from a cloud storage system), by the processor, mobile health data from one or more mobile health devices of the user, the mobile health data comprising one or more measurements recorded by the one or more mobile health devices; and automatically determining, by the processor, the feedback recommendation based on the genotyping data for the user (e.g. the personal genetic profile assessment) and the received mobile health data.

In certain embodiments, the mobile health data comprises one or more measurements selected from the group consisting of: (i) average and/or aggregate calorie intake over a period of time; (ii) a glucose measurement; (iii) a physical activity level metric (e.g. an average and/or aggregate number of steps taken over a period of time; e.g. a recorded workout); and (iv) a brain wave measurement (e.g. an EEG measurement).

In another aspect, the invention is directed to a system for automatically providing genetically tailored notifications to one or more mobile health devices of an individual based on an assessment of the individual's genetic profile, the system comprising: a processor; and a non-transitory computer readable medium having instructions stored thereon, wherein the instructions, when executed by the processor, cause the processor to: receive (and/or access) genotyping data (e.g., a personal genetic profile assessment) corresponding to a biological sample of a user (e.g., one or more genotyping measurements of one or more SNPs, each SNP associated with one or more genes); automatically determine a feedback recommendation (e.g. a recommendation to reduce physical activity level; e.g. a recommended physical activity level; e.g. a recommended number of meditation sessions) based on the genotyping data for the user (e.g. the personal genetic profile assessment); and cause creation of, for presentation to the user (e.g. for presentation on a user mobile health device; e.g. for presentation on a user computing device), a notification (e.g. a graphically rendered notification comprising one or more icons and/or alphanumeric strings displayed on a user mobile health device; e.g. an auditory notification (e.g. an alarm; e.g. an audio message); e.g. a haptic cue (e.g. a vibration), e.g. any combination of a graphically rendered notification, an auditory notification and a haptic cue) corresponding to the feedback recommendation.

In certain embodiments, the instructions further cause the processor to: receive (and/or access) (e.g. via a network; e.g. from a cloud storage system) mobile health data from one or more mobile health devices of the user, the mobile health data comprising one or more measurements recorded by the one or more mobile health devices; and automatically determine the feedback recommendation based on the genotyping data for the user (e.g. the personal genetic profile assessment) and the received mobile health data.

In certain embodiments, the mobile health data comprises one or more measurements selected from the group consisting of: (i) average and/or aggregate calorie intake over a period of time; (ii) a glucose measurement; (iii) a physical activity level metric (e.g. an average and/or aggregate number of steps taken over a period of time; e.g. a recorded workout); and (iv) a brain wave measurement (e.g. an EEG measurement).

In another aspect, the invention is directed to a method for automatically identifying and providing for graphical rendering and presentation to a user via a graphical user interface (GUI), portions of their personal genetic profile assessment as a reward based on visits to various participating merchants, the method comprising: (a) receiving (e.g., and/or accessing), by a processor of a computing device, data corresponding to an indication of a user visit to a specific participating merchant (e.g., an alert that the user is in a physical store of the specific participating merchant; e.g., an alert that the user is making a purchase at the specific participating merchant; e.g., an alert that the user has made a purchase at the specific participating merchant); (b) accessing, by the processor, a merchant database comprising a plurality of merchant identifiers, each of which identifies a particular participating merchant, wherein each merchant identifier is associated with one or more unlockable set(s), each of which comprises identifiers of one or more SNPs and/or genes; (c) matching, by the processor, the specific participating merchant to a merchant identifier within the merchant database; (d) selecting, by the processor, at least one of the one or more unlockable set(s) associated with the matching merchant identifier; (e) accessing, by the processor, genotyping data for the user (e.g., a personal genetic profile assessment), wherein: the genotyping data comprises results of genotyping measurements of a plurality of SNPs for the user; at least a portion of the results are initially locked; and at least a portion of the initially locked results correspond to the selected unlockable set; and (f) unlocking, by the processor, the portion of the initially locked results corresponding to the selected unlockable set (e.g., and, following the unlocking, issuing to the user a notification of the unlocking).

In certain embodiments, the method comprises causing, by the processor, graphical rendering of, for presentation to the user (e.g., for presentation on a user mobile computing device), a graphical representation of the unlocked results.

In certain embodiments, the method comprises issuing, by the processor, a deal notification to the user (e.g., an email; e.g.; a text message; e.g., a push notification), the deal notification comprising an identification of the specific participating merchant and an identification of the one or more unlockable sets associated with the merchant identifier that identifies the specific participating merchant.

In certain embodiments, the method comprises: receiving, by the processor, user location data that identifies a location of the user (e.g., based on GPS data from a smart phone of the user); determining, by the processor, using the user location data, the user to be in proximity to (e.g., within a predefined distance of) a physical location (e.g., a store) of the specific participating merchant; and responsive to determining that the user is in proximity to a physical location of the specific participating merchant, issuing, by the processor, the deal notification to the user.

In certain embodiments, each of at least a portion (e.g., one or more) of the one or more unlockable set(s) associated with the merchant identifier matching the specific participating merchant is associated with a set of criteria for unlocking the set and the method comprises issuing, by the processor, to the user, a notification comprising, for at least one unlockable set, the associated criteria for unlocking.

In certain embodiments, step (d) comprises determining that the data corresponding to an indication of a user visit to a specific participating merchant satisfies a set of criteria associated with the selected unlockable set.

In another aspect, the invention is directed to a method for creating and storing, in a merchant database, genetic profile assessment rewards associated with participating merchants, the method comprising: (a) presenting, by a processor of a computing device, a graphical user interface element (e.g., widget) for creation of a genetic profile assessment reward associated with a specific participating merchant; (b) receiving, by the processor, via the GUI element, a merchant identifier that identifies the specific participating merchant; (c) receiving, by the processor, via the GUI element, a selection of a set of identifiers of one or more SNPs and or genes to create an unlockable set that identifies a portion of genotyping data results to be unlocked via the reward; (d) associating, by the processor, the merchant identifier with the created unlockable set; and (e) storing, by the processor, the merchant identifier and association with the unlockable set in the merchant database, for further updating and/or accessing (e.g., in order to unlock the data).

In certain embodiments, the method comprises: receiving, by the processor, via the GUI element, a set of criteria for unlocking results; associating, by the processor, the set of criteria with the unlockable set; and storing, by the processor, the association with the unlockable set in the merchant database.

In another aspect, the invention is directed to a system for automatically identifying and providing for graphical rendering and presentation to a user via a graphical user interface (GUI), portions of their personal genetic profile assessment as a reward based on visits to various participating merchants, the system comprising: a processor of a computing device; and a memory having instructions stored thereon, wherein the instructions, when executed by the processor, cause the processor to: (a) receive (e.g., and/or access) data corresponding to an indication of a user visit to a specific participating merchant (e.g., an alert that the user is in a physical store of the specific participating merchant; e.g., an alert that the user is making a purchase at the specific participating merchant; e.g., an alert that the user has made a purchase at the specific participating merchant); (b) access a merchant database comprising a plurality of merchant identifiers, each of which identifies a particular participating merchant, wherein each merchant identifier is associated with one or more unlockable set(s), each of which comprises identifiers of one or more SNPs and/or genes; (c) match the specific participating merchant to a merchant identifier within the merchant database; (d) select at least one of the one or more unlockable set(s) associated with the matching merchant identifier; (e) access genotyping data for the user (e.g., a personal genetic profile assessment), wherein: the genotyping data comprises results of genotyping measurements of a plurality of SNPs for the user; at least a portion of the results are initially locked; and at least a portion of the initially locked results correspond to the selected unlockable set; and (f) unlock the portion of the initially locked results corresponding to the selected unlockable set (e.g., and, following the unlocking, issue to the user a notification of the unlocking).

In certain embodiments, the instructions cause the processor to cause graphical rendering of, for presentation to the user (e.g., for presentation on a user mobile computing device), a graphical representation of the unlocked results.

In certain embodiments, the instructions cause the processor to issue a deal notification to the user (e.g., an email; e.g.; a text message; e.g., a push notification), the deal notification comprising an identification of the specific participating merchant and an identification of the one or more unlockable sets associated with the merchant identifier that identifies the specific participating merchant.

In certain embodiments, the instructions cause the processor to: receive user location data that identifies a location of the user (e.g., based on GPS data from a smart phone of the user); determine, using the user location data, the user to be in proximity to (e.g., within a predefined distance of) a physical location (e.g., a store) of the specific participating merchant; and issue, responsive to determining that the user is in proximity to a physical location of the specific participating merchant, the deal notification to the user.

In certain embodiments, each of at least a portion (e.g., one or more) of the one or more unlockable set(s) associated with the merchant identifier matching the specific participating merchant is associated with a set of criteria for unlocking the set and the instructions cause the processor to issue to the user a notification comprising, for at least one unlockable set, the associated criteria for unlocking.

In certain embodiments, at step (d) the instructions cause the processor to determine that the data corresponding to an indication of a user visit to a specific participating merchant satisfies a set of criteria associated with the selected unlockable set.

In another aspect, the invention is directed to a system for creating and storing, in a merchant database, genetic profile assessment rewards associated with participating merchants, the system comprising: a processor of a computing device; and a memory having instructions stored thereon, wherein the instructions, when executed by the processor, cause the processor to: (a) presenting, by a processor of a computing device, a graphical user interface element (e.g., widget) for creation of a genetic profile assessment reward associated with a specific participating merchant; (b) receiving, by the processor, via the GUI element, a merchant identifier that identifies the specific participating merchant; (c) receiving, by the processor, via the GUI element, a selection of a set of identifiers of one or more SNPs and or genes to create an unlockable set that identifies a portion of genotyping data results to be unlocked via the reward; (d) associating, by the processor, the merchant identifier with the created unlockable set; and (e) storing, by the processor, the merchant identifier and association with the unlockable set in the merchant database, for further updating and/or accessing (e.g., in order to unlock the data).

In certain embodiments, the instructions cause the processor to: receive, via the GUI element, a set of criteria for unlocking results; associate the set of criteria with the unlockable set; and store the association with the unlockable set in the merchant database.

### Definitions

In order for the present disclosure to be more readily understood, certain terms used herein are defined below. Additional definitions for the following terms and other terms may be set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Genotyping data: As used herein, the term “genotyping data” refers to data obtained from measurements of a genotype. Measurements of a genotype performed on a biological sample identify the particular nucleotide(s) (also referred to as “bases”) that is/are incorporated at one or more particular positions in genetic material extracted from the biological sample. Accordingly, genotyping measurements for a particular individual are measurements performed on a biological sample of from the individual, and which identify the particular nucleotides present at one or more specific positions within their genome.

In certain embodiments, genotyping data describes an individual's phenotype. Genotyping data may be measurements of particular genes (e.g., portions of an individual's genetic sequence, e.g., DNA sequence), SNPs, or variants of SNPs. For example, a genotyping measurement of a particular SNP for an individual identifies the particular variant of that SNP that the individual has. A genotyping measurement of a particular gene for an individual identifies the particular nucleotides that are present at one or more locations within and/or in proximity to the gene for the individual. For example, genotyping measurements of a particular gene may identify the particular variants of one or more SNPs associated with a particular gene.

In certain embodiments, genotyping data is obtained from a multi-gene panel. In certain embodiments, genotyping data is obtained from assays (e.g., TaqMan™ assays) that detect one or more specific variants of specific SNPs. In certain embodiments, genotyping data is obtained from genetic sequencing measurements. In certain embodiments, genotyping data is generated in response to a purchase or request by an individual. In certain embodiments, genotyping data comprises data for a portion of a genotype (e.g., of an individual). In certain embodiments, genotyping data comprises all available measurements of a genotype (e.g., of an individual).

Supplement: As user herein, the term “supplement” refers to a product ingested, consumed, and/or applied by a user in order to do at least one of: enhance wellbeing, improve performance or function, and counteract effects of a chronic condition. A supplement may be a vitamin, multivitamin, mineral, dietary supplement, herb, botanical, concentrate, metabolite, extract, amino acid, over-the-counter medication, prescription medication, topical product, or health/treatment regimen or program. In certain embodiments, a supplement is to be taken on a recurring basis (e.g., daily or twice daily) by a user for a period of time. A period of time may be an ongoing basis with no pre-determined cessation period. In certain embodiments, a supplement is a program or regimen that a user can enroll in or purchase access to. For example, a supplement may be a behavioral program such as a focus program or a personalized fitness plan (e.g., for use in home exercise).

Variant: As used herein, the terms “variant” refers to a specific variation of a specific SNP occurring in the genetic material of a population. In certain embodiments, a variant is a specific combination of a first allele of a first copy of an individual's genetic material (e.g. corresponding to an individual's paternal DNA) and a second allele of a second copy of an individual's genetic material (e.g. corresponding to an individual's maternal DNA), as occurs in diploid organisms (e.g. humans).

Qualifier: As used herein, the term “qualifier” refers to a classification (e.g. a label) of a particular variant of a given SNP. The qualifier associated with a given variant is the particular classification (e.g. label) of that variant. For example, a given variant may be associated with a particular qualifier of a predefined set of possible qualifiers. For example, a given variant may be associated with a qualifier selected from a group of labels such as “Adapt,” “Normal,” and “Gifted.” In certain embodiments, for a given variant of a given SNP, a qualifier corresponds to a classification of the given variant based on (i) the prevalence of the given variant within a population (e.g. if the variant is common, e.g. if the variant is rare) and/or (ii) a health-related trait associated with the variant. For example, a common variant may be associated with the qualifier “Normal”. A rare variant that confers a disadvantageous phenotype, such as a predisposition to high cholesterol, may be associated with the qualifier “Adapt” (e.g. classified as rare and disadvantageous). A rare variant that confers an advantageous phenotype, such as a predisposition to lower cholesterol, may be associated with the qualifier “Gifted” (e.g. accordingly, the variant is classified as rare and advantageous).

Variant object: As used herein, the term “variant object” refers to a data structure corresponding to (e.g. that is used to represent) a specific variant of a physical SNP and/or gene within a given genome (e.g., the genome of a human).

SNP object: As used herein, the term “SNP object” refers to a data structure corresponding to (e.g. that is used to represent) a specific single nucleotide polymorphism (SNP). In certain embodiments, a SNP object comprises a SNP reference that identifies the specific SNP to which the SNP object corresponds. The SNP reference may be an alphanumeric code such as an accepted name of the SNP or other identifying mark or label capable of being stored electronically. The SNP reference may be an alphanumeric code such as a National Center for Biotechnology Information (NCBI) database reference number.

Gene object: As used herein, the term “gene object” refers to a data structure corresponding to (e.g. that is used to represent) a specific physical gene within a given genome (e.g. the human genome).

Category: As used herein, the term “category” refers to a data structure corresponding to (e.g. that is used to represent) a particular health-related trait or characteristic.

Product, Genetic Profile Product, Personal Genetic Profile Product: As used herein, the terms “product,” “genetic profile product,” and “personal genetic profile product,” refer to a data structure corresponding to (e.g. that is used to represent) a general class of health-related traits and/or characteristics. In certain embodiments, a product is associated with one or more categories that correspond to health-related traits and characteristics related to the general class of health-related traits and characteristics to which the product corresponds.

Personal Genetic Profile Assessment: As used herein, the term “personal genetic profile assessment” refers to a data structure (e.g., a hierarchy of data structures) corresponding to (e.g. that is used to represent) the phenotype of a user for one or more general classes of health-related traits and/or characteristics. In certain embodiments, a personal genetic profile assessment of a user is generated by associating genotyping data of the user with premade (i.e., stored) generic personal genetic profile products. In certain embodiments, a user's personal genetic profile assessment is viewed using an assessment graphical user interface (“assessment GUI”) on a computing device (e.g., a smartphone).

Developer: As used herein, the term “developer” refers to a person, company, or organization that uses a graphical user interface to create data structures. In certain embodiments, a developer also genotypes a biological sample in response to an assessment corresponding to a product being purchased or made accessible to an individual.

User: As used herein, the term “user” refers to a person who uses an assessment graphical user interface in order to view information about a genome. The user may supply one or more biological samples to be genotyped in order for a personal genetic profile assessment to be formed. The user may purchase or be given access to one or more products in order to view a personal genetic profile assessment. The user may purchase one or more supplements from a list of purchase recommendations provided in the graphical user interface that are based on the user's personal genetic profile assessment. The terms “user” and “individual” are used interchangeably herein.

Graphical Control Element: As used herein, the term “graphical control element” refers to an element of a graphical user interface element that may be used to provide user and/or individual input. A graphical control element may be a textbox, dropdown list, radio button, data field, checkbox, button (e.g., selectable icon), list box, or slider.

Associate, Associated with: As used herein, the terms “associate,” and “associated with,” as in a first data structure is associated with a second data structure, refer to a computer representation of an association between two data structures or data elements that is stored electronically (e.g. in computer memory).

Provide: As used herein, the term “provide”, as in “providing data”, refers to a process for passing data in between different software applications, modules, systems, and/or databases. In certain embodiments, providing data comprises the execution of instructions by a process to transfer data in between software applications, or in between different modules of the same software application. In certain embodiments a software application may provide data to another application in the form of a file. In certain embodiments an application may provide data to another application on the same processor. In certain embodiments standard protocols may be used to provide data to applications on different resources. In certain embodiments a module in a software application may provide data to another module by passing arguments to that module.

Mobile health device: As used herein, the term “mobile health device”, refers to any one of a variety of mobile devices that a user uses to record data such as biological/physical measurements as well as activity data about activities they perform related to physical health. Data recorded by a mobile health device is referred to herein as “mobile health data”. In certain embodiments, mobile health data includes measurements such as weight, glucose levels, recorded calorie intake, as well as data about physical activities such as an average or aggregate number of steps taken over a given period of time, recorded workouts (e.g. as recorded by a fitness monitoring software app operating on a mobile health device), sleep quality data, and brain wave data (e.g. EEG measurements). In certain embodiments, mobile health devices are network connected devices, such that mobile health data recorded by a given mobile health device can be accessed and/or received by a processor (e.g. of another computing device) over a network. In certain embodiments, mobile health devices include activity tracking devices (e.g. devices for monitoring exercise, steps, pulse rate, sleep, eating, or other activity), mobile phones (e.g. smartphones), tablet computers, brain activity monitoring devices (e.g., devices for monitoring mental focus, alertness, mental stress, relaxation, sleep, or the like), connected home devices (e.g. a network connected scale).

### BRIEF DESCRIPTION OF THE DRAWINGS

Drawings are presented herein for illustration purposes, not for limitation. The foregoing and other objects, aspects, features, and advantages of the invention will become more apparent and may be better understood by referring to the following description taken in conjunction with the accompanying drawings, in which:

FIG. 1 is a block diagram illustrating associations between different data structures in personal genetic profile products, according to an illustrative embodiment of the invention;

FIG. 2 is a block diagram showing an organizational hierarchy of a personal genetic profile product, according to an illustrative embodiment of the invention;

FIG. 3 is a block diagram showing a process for creating a personal genetic profile assessment, according to an illustrative embodiment of the invention;

FIG. 4 is a portion of a text file comprising genotyping data, according to an illustrative embodiment of the invention;

FIG. 5 is a block diagram of a method for automatically identifying, and providing for graphical rendering and presentation to a user via graphical user interface (GUI), a purchase recommendation based on an assessment of an individual's genetic profile, according to an illustrative embodiment of the invention;

FIG. 6 is a block diagram of a method for linking supplement purchase recommendations with personal genetic profile products, according to an illustrative embodiment of the invention;

FIG. 7 is a block diagram of an example network environment for use in the methods and systems described herein, according to an illustrative embodiment of the invention; and

FIG. 8 is a block diagram of an example computing device and an example mobile computing device, for use in illustrative embodiments of the invention.

FIG. 9 is a screenshot of a portion of a graphical user interface for presenting to a user an assessment of their genetic profile, according to an illustrative embodiment of the invention.

FIG. 10 is a screenshot of a portion of a graphical user interface for presenting to a user an assessment of their genetic profile, according to an illustrative embodiment of the invention.

FIG. 11A is a screenshot of a portion of a graphical user interface for presenting to a user an assessment of their genetic profile showing a view that displays a specific measurement outcome for a specific SNP associated with a specific gene and a description of the measurement outcome, according to an illustrative embodiment of the invention.

FIG. 11B is a screenshot of a portion of a graphical user interface for presenting to a user an assessment of their genetic profile showing a view that displays a description of a specific measurement outcome for a specific SNP associated with a specific gene, according to an illustrative embodiment of the invention.

FIG. 12A is a block flow diagram of a process for automatically identifying and providing for graphical rendering and presentation to a user via a graphical user interface (GUI), portions of their personal genetic profile assessment based on visits to various participating merchants, according to an illustrative embodiment.

FIG. 12B is a block flow diagram of a process for creating and storing, in a merchant database, genetic profile assessment rewards associated with participating merchants, according to an illustrative embodiment.

FIG. 13 is a block flow diagram of a process for determining and creating, for presentation to a user, feedback notifications based on genotyping data for the user.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### DETAILED DESCRIPTION OF THE INVENTION

It is contemplated that systems, architectures, devices, methods, and processes of the claimed invention encompass variations and adaptations developed using information from the embodiments described herein. Adaptation and/or modification of the systems, architectures, devices, methods, and processes described herein may be performed, as contemplated by this description.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim. Documents are incorporated herein by reference as noted. Where there is any discrepancy in the meaning of a particular term, the meaning provided in the Definition section above is controlling.

Headers are provided for the convenience of the reader and are not intended to be limiting with respect to the claimed subject matter.

Presented herein are systems and methods for automatically identifying and recommending purchases (e.g., in-app purchases) to a user based on the user's personal genetic profile. In certain embodiments, offers for such purchases are conveniently presented in the same software application (e.g., smartphone app or other computing device application) in which a user securely accesses his or her personalized genetic profile test results. Also presented herein are systems and methods for computer application developers to customize apps for presentation of recommended purchases based on a user's personal genetic profile.

In certain embodiments, a user's personal genetic profile is graphically rendered for presentation to a user (e.g., via smartphone app). The profile may be limited to, and/or organized according to, one or more different products of health-related traits and characteristics. Examples of products include (i) nutritional characteristics (e.g., the way in which an individual's body processes different foods and nutrients), (ii) skin health, (iii) physical fitness, and (iv) personal behavior tendencies (e.g., empathy, risk of addiction, and tolerance for stress and pain). Depending on a particular user's personal genetic profile results in a given product, supplements may be identified for presentation to the user as a recommended purchase, in light of the user's profile results.

In certain embodiments, genotyping data determined from a biological sample provided by an individual is used as a basis for identification of supplements that are relevant for the individual due to their particular genetic makeup. Different individuals have different variants of particular SNPs, each SNP associated with one or more particular genes. For a given SNP corresponding to a given gene, the particular variant that an individual has influences a specific health-related trait. These health-related traits may be related to an individual's propensity to gaining weight, their ability to process certain vitamins, longevity (e.g., rate at which they age), joint and muscle health, endurance, and lean body mass, and skin health, for example. In certain embodiments, recommendations for supplements are made to individuals based on the particular health-related phenotype of each individual.

A collection of genotyping data (e.g., corresponding to biological traits) of an individual may be stored and organized in a personal genetic profile assessment. A personal genetic profile assessment is a complex data structure that associates related sub-structures that correspond to genes and SNPs. A personal genetic profile assessment is used to populate an assessment GUI that is used by a user to view and navigate information about his or her phenotype. A user may also access purchase recommendations for supplements, recommended based on health-related traits of the user, using the assessment GUI or external links embedded in the assessment GUI for the user.

### Storage and Presentation of Personal Genetic Profile Assessments

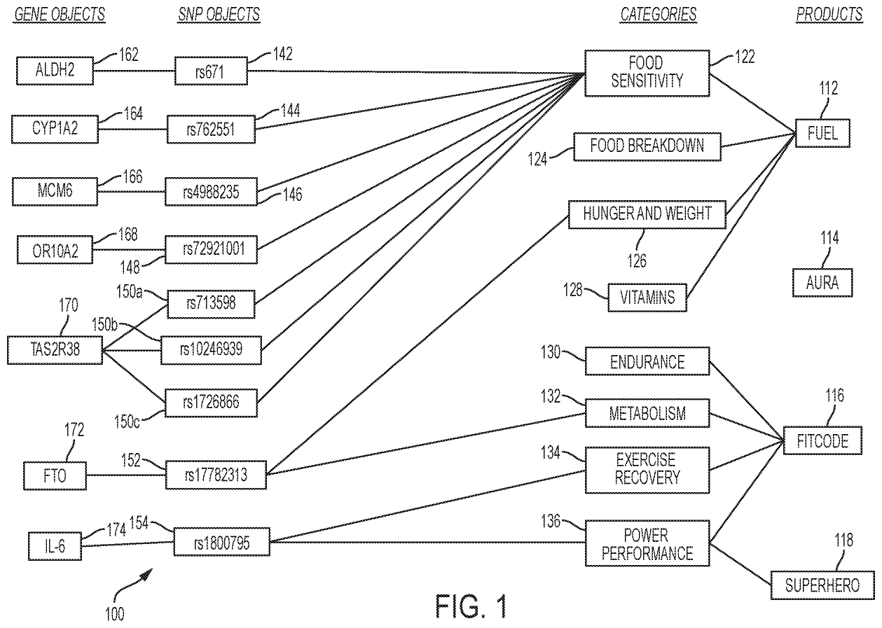

Turning to FIG. 1, in certain embodiments, in order to provide an individual not only with their personal genetic profile assessment, but also convey information related to the particular traits and characteristics that are influenced by the specific SNP variants present in their genetic material in an organized and intuitive fashion, the systems and methods described herein provide a framework comprising an intuitive hierarchical organization of data structures. The framework provides for storing relationships (e.g. associations) between particular SNPs, biological traits and characteristics, and general classes of such traits and characteristics, based on the specific traits that each particular SNP influences.

In certain embodiments, a first (e.g., top level) class of data structures, referred to herein as products, are used to represent different general classes of health-related traits and characteristics. In certain embodiments, a product data structure corresponds to a particular assessment ordered (e.g., purchased by the individual), in which unique versions of genes and/or SNPs that an individual has that influence the particular general class of health-related traits and characteristics that the corresponding product represents are identified (e.g., via genotyping measurements).

In certain embodiments, each product has a name (e.g. a product data structure comprises a name (e.g. text data representing the name)) that provides a convenient, and memorable way to refer to the product. For example, a particular product 112 (e.g. named “FUEL™”) is used to represent a class of traits corresponding to the way in which an individual's body processes different foods and nutrients. Another product 114 (e.g. named “AURA™”) is used to represent a class of traits corresponding to skin health. Another product 116 (e.g. named “FITCODE™”) is used to represent a class of traits corresponding to physical fitness. Another product 118 (e.g. named “SUPERHERO™”) is used to represent a class of traits corresponding to physical and intellectual performance. In certain embodiments, a name of a product is the same as the name under which a particular assessment is offered for sale. For example, assessments FUEL™, FITCODE™, AURA™, and SUPERHERO™ are offered for sale by Orgi3n, Inc. of Boston Mass.

In certain embodiments, each product is in turn associated with one or more of a second class of data structures, referred to as categories. In certain embodiments, each category corresponds to a particular health-related trait or characteristic (e.g. food sensitivity, food breakdown, hunger and weight, vitamins, skin uv sensitivity, endurance, metabolism, joint health, muscle strength, intelligence). In certain embodiments, the categories with which a particular product is associated each correspond to different health-related traits or characteristics that are related to the general class of health-related traits or characteristics to which the particular product corresponds (e.g. the general class of health-related traits or characteristics that the product represents). As with products, in certain embodiments, each category has a name (e.g. a category data structure comprises a name (e.g. text data representing the name)) that provides a convenient, and memorable way to refer to the category.

In turn, each category is associated with one or more SNP objects, each SNP object corresponding to a specific SNP. Each SNP object associated with a particular category corresponds to a specific SNP that influences a specific health related trait that relates to the trait or characteristic to which the particular category corresponds. Each SNP object may identify the specific SNP to which it corresponds via a SNP reference that the SNP object comprises. The SNP reference may be an alphanumeric code such as an accepted name of the SNP or other identifying mark or label capable of being stored electronically. The SNP reference may be an alphanumeric code such as a National Center for Biotechnology Information (NCBI) database reference number.

For example, the schematic of FIG. 1 shows an example of series of products, categories, and SNP objects that are associated with each other. Associated gene objects, to be described in the following, are also shown. The different products and categories are identified by their particular names, and the SNP objects each are identified by a respective SNP reference each comprises. In the example of FIG. 1, the SNP references are NCBI database reference numbers.

The “FUEL™” product 112 is associated with categories such as “Food Sensitivity” 122, “Food Breakdown” 124, “Hunger and Weight” 126, and “Vitamins” 128. Several SNP objects corresponding to specific SNPs that influence characteristics related to an individual's sensitivity to different types of foods, and, accordingly, are associated with the “Food Sensitivity” category 122 are shown. In FIG. 1, the lines connecting the SNP objects to different categories indicate the association of each particular SNP object with one or more different categories. The associations may be direct associations or indirect associations (i.e., through mutual association with an intermediate data structure not shown).

For example, SNP object 132 corresponds to the rs671 SNP, which influences the manner in which an individual processes alcohol. In particular, depending on the particular variant of the rs671 SNP that an individual has, the individual may process alcohol normally, or be impaired in their ability to process alcohol, and likely suffer from adverse effects resulting from alcohol consumption, such as flushing, headaches, fatigue, and sickness. Accordingly, providing individuals with knowledge of the particular variant of the rs671 SNP they possess may allow them to modify their behavior accordingly, for example, by being mindful of the amounts of alcohol that they consume (e.g. on a regular basis, e.g. in social settings).

Other SNP objects corresponding to SNPs that influence food sensitivity related characteristics, and, accordingly, are associated with the “Food Sensitivity” category 222 are shown. For example, SNP object 144 corresponds to the rs762551 SNP that influences caffeine metabolism, SNP object 146 corresponds to the rs4988235 SNP that influences lactose intolerance, and SNP object 148 corresponds to the rs72921001 SNP that influences an aversion to the herb cilantro (e.g. depending on the particular variant of this SNP that an individual has, they may either perceive cilantro as pleasant tasting or bitter and soap-like in taste).

In certain examples, multiple SNPs are associated with a particular characteristic and, accordingly, the SNP objects to which they correspond may be grouped together. For example, three SNPS—rs713598 (corresponding to SNP object 150a), rs10246939 (corresponding to SNP object 150b), and rs1726866 (corresponding to SNP object 150c), —influence the sensitivity of individuals to bitter tasting foods (e.g. cabbage, broccoli, cauliflower, kale, brussel sprouts, and collard greens), and, accordingly, their enjoyment of or aversion to such foods.

SNPs correspond to specific locations within or nearby (e.g., a SNP may occur in a promotor region that influences transcription of a particular gene, e.g., a SNP may occur within 5 kb upstream or downstream of a particular gene, e.g., a SNP may occur within 100 kb upstream or downstream of a particular gene, e.g., a SNP may occur within 500 kb upstream or downstream of a particular gene, e.g., a SNP may occur within 1 Mb upstream or downstream of a particular gene) genes in an individual's genetic material. Accordingly, in certain embodiments, as shown in FIG. 1, each SNP object is associated with a gene object that corresponds to the particular gene within or nearby to which the SNP to which the SNP object corresponds is present. For example, the rs671 SNP corresponds to a location within the ALDH2 gene; the rs762551 SNP corresponds to a location within the CYP1A2 gene, the rs4988235 SNP occurs within the MCM6 gene, and the rs72921001 SNP occurs within the OR10A2 gene. Accordingly, SNP object 142 (corresponding to the rs671 SNP) is associated with gene object 162 (corresponding to the ALDH2 gene). Similarly, SNP object 144 (corresponding to the rs762551 SNP) is associated with gene object 162 (corresponding to the CYP1A2 gene), SNP object 146 (corresponding to the rs4988235 SNP) is associated with gene object 166 (corresponding to the MCM6 gene) and SNP object 148 (corresponding to the rs72921001 SNP) is associated with gene object 168 (corresponding to the OR10A2 gene).

Other SNPs objects correspond to SNPs that are nearby particular genes of interest and thereby influence characteristics associated with expression of the gene. For example, rs12696304 is a SNP that lies 1.5 kb downstream from the TERC gene, and influences biological aging associated with the TERC gene. Accordingly, in one example, a SNP object corresponding to the rs12696304 SNP is associated a gene object corresponding to the TERC gene.

In certain embodiments, multiple SNPs of interest occur within a single gene. For example, the three SNPs related to bitter taste—rs713598, rs10246939, and rs1726866—occur within the TAS2R38 gene. Accordingly, SNP objects 150a, 150b, and 150c, which correspond to the rs713598, rs10246939, and rs1726866 SNPs, respectively, are all associated with a gene object 170 corresponding to the TAS2R38 gene.

In certain embodiments, different products correspond to different general classes of health-related traits and characteristics. For example, products may be based on particular organs (e.g. product 114, named “AURA™”, is related to skin health), or particular habits, activities, or bodily functions. For example, food related biological characteristics and traits may be covered by a single products or a plurality of products. A single product or a plurality of products may be based on learning and brain function characteristics and traits. A single product or a plurality of products may be based on physical fitness (e.g., cardiovascular strength, agility, flexibility, muscular strength).

For example, as shown in FIG. 1, another product 116 (e.g. named “FITCODE™”), relates to a general class of physical fitness related traits, and, accordingly, comprises categories associated with endurance 130 (“Endurance”), metabolism 132 (“Metabolism”), the ability of an individual to recover effectively following exercises 134 (“Exercise Recovery”), and cardiovascular fitness and skeletal muscle makeup 136 (“Power Performance”).

In certain embodiments, a particular SNP object is associated with two or more categories. For example, the rs17782313 SNP, occurring in the FTO gene, influences an individual's appetite. Accordingly, as shown in FIG. 1, the SNP object 152 corresponding to the rs17782313 SNP is associated with both the “Hunger and Weight” category 126 of the “FUEL™” product, and the “Metabolism” category 132 of the “FITCODE™” product. SNP object 152 is also associated with gene object 172, reflecting the fact that the rs17782313 SNP occurs in the FTO gene. In certain embodiments, as with the rs17782313 SNP object, each of a first category and a second category with which a particular SNP object is associated are associated with a different product. In certain embodiments, a particular SNP object is associated with a first category and a second category, and both the first category and the second category are associated with the same product.

For example, the SNP object 154 corresponding to the rs1800795 SNP of the IL-6 gene (accordingly, SNP object 154 is associated with gene object 174, which corresponds to the IL-6 gene) is associated with the “Exercise Recovery” category 134 and the “Power Performance” category 136, both of which are associated with the “FITCODE™” product 116. In addition, in certain embodiments, a category is associated with two or more products. For example, the “Power Performance” category 136 is associated with the “FITCODE™” product 116, as well as the “SUPERHERO™” product 118, which provides an assessment of a general class of traits related to physical and intellectual performance.

In certain embodiments the hierarchical organization of product, category, SNP object, gene object, and variant object data structures serves as a flexible template that facilitates both the rapid creation of individual personal genetic profile assessments from genotyping measurements taken from a plurality of individuals, and the presentation of an individual's personal genetic profile assessment. In particular, an individual may purchase assessments corresponding to different products, in order to gain insight into the manner in which their personal genome influences the different general classes of health-related traits and characteristics to which each different product corresponds. Accordingly, an individual's personal genetic profile assessment corresponding to one or more products comprises, for each specific SNP associated with each category that is associated with each of the one or more products, an identification of the particular variant of the specific SNP that the individual has. Typically, the identification is obtained via one or more genotyping measurements performed on a biological sample taken from the individual (e.g. a blood sample, e.g. a cheek swab sample, e.g. a saliva sample, e.g. a hair sample, e.g. hair follicle cells).

In certain embodiments, an individual may purchase a first assessment corresponding to a first product, and provide a biological sample for genotyping. The individual's biological sample may be stored (e.g. cryogenically frozen). After a period of time, the individual may choose to purchase additional assessments corresponding to other products, and the individual's previously stored biological sample may be taken from storage for additional genotyping measurements of the additional SNPs that are associated with the new products. Moreover, in certain embodiments, additional new products may be created over time, and new assessments corresponding to new products offered to and purchased by individuals. In certain embodiments, as new information related to the influence of new and/or existing SNPs on different specific health related characteristics is elucidated, new SNP objects and gene objects may be created, and new associations between them and new or existing categories and/or products established. In certain embodiments, existing personal genetic profile assessments of individuals are automatically updated to reflect new information.

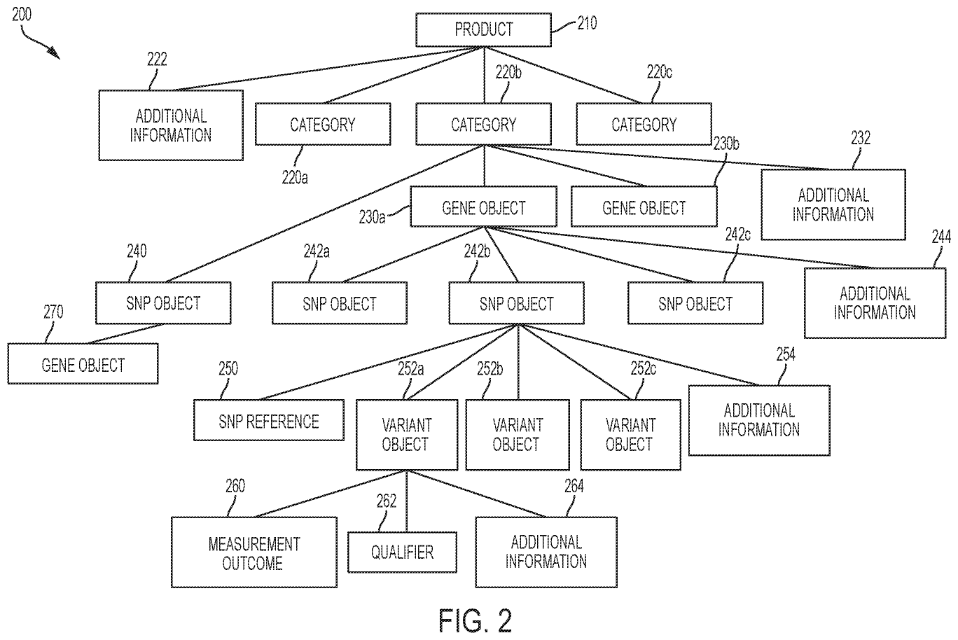

In certain embodiments, in order to facilitate the creation and presentation of individual personal genetic profile assessments (e.g. corresponding to one or more different products) based on the framework described above, the product, category, SNP object, and gene object data structures described herein are created and associated as a generic hierarchy of data structures to later be associated with the genotyping data of an individual. FIG. 2 is a block diagram of a hierarchy of data structures 200 of an example genetic profile product. In certain embodiments, a developer creates and stores one or more generic hierarchies of data structures in accordance with FIG. 2 that define one or more products that may be purchased and/or accessed by an individual. The hierarchies of data structures are generic in that they contain no personal information for any one individual, but instead define the collection of genes, SNPs, and variants that have relevance to the biological characteristics and/or traits that are encompassed by a product.

An exemplary data structure of each type is shown to be associated with sub-data structures in FIG. 2 in order to simplify presentation of the figure. It is understood that data structures may be associated to any number of other data structures in the hierarchy if the association is consistent with the associations shown in FIG. 2. For example, category 220b is shown to be associated with gene objects 230a-b while category 220c may be associated with one or more gene objects and/or SNP objects, but any such associations are not shown. In some embodiments, data structures may be created without also forming associations between other structures of relevant types. For example, unassociated or partially associated data structures may be created for planning purposes such as during product or category development (e.g., category 220a has no associations yet because its scope has not been determined yet by the user). For example, unassociated or partially associated data structures may be created to allow genotyping data to be associated with relevant gene objects or SNP objects in order to retain the data in a ready to use format in the event that the gene objects and/or SNP objects are later associated with one or more categories.

Referring now to FIG. 2, product 210 comprises three categories 220a-c and additional information 222. Additional information 222 may be a name of the product, an icon associated with the product, and/or a description of the product. Category 220b comprises two gene objects 230a-b, one SNP object 240, and additional information 232. Additional information 232 may comprise a name of the category, a background image associated with the category, an icon associated with the category, a category order identifier, and/or a description of the category. SNP object 240 is associated with gene object 270. Gene object 230a is associated to three SNP objects 242a-c. Categories may be associated directly to SNP objects, such as category 220b is associated with SNP object 240, or they may be associated indirectly such as SNP objects 242a-c are associated to category 220b via gene object 230a. The ability to form associations indirectly allows all SNP objects associated with a particular gene object to be associated with a category by forming a single association in cases where all SNP objects of a particular gene are relevant to a particular category. The ability to form associations directly allows a particular SNP object to be associated with a category without also forming an association with all other SNP objects associated with the gene object associated with the particular SNP object in cases where only one or a subset of SNP objects of a particular gene object are relevant to a category.

Gene object 230a is also associated with additional information 244. Additional information 244 may comprise one or more data structures comprising information such as a unique gene identifier that corresponds gene object 230a to a specific physical gene and descriptive information about the corresponding gene. The gene identifier may be an alphanumeric code such as an accepted name of the gene or other identifying mark or label capable of being stored electronically. Additional information may be stored as a single data structure or a plurality of data structures.

SNP object 242b is associated with SNP reference 250, and additional information 254. SNP reference 250 is a unique identifier of the SNP that corresponds the SNP object to a specific physical SNP. The SNP reference may be an alphanumeric code such as an accepted name of the gene or other identifying mark or label capable of being stored electronically. The SNP reference may be an alphanumeric code such as a National Center for Biotechnology Information (NCBI) database reference number. Additional information 254 may comprise one or more data structures with other descriptive information about the corresponding SNP.

Variants of a particular SNP can be represented within a corresponding SNP object using various combinations of data elements such as a measurement outcomes, and qualifiers. For example, a particular variant of a SNP can be identified by a measurement outcome, which is an identifier, such as an alphanumeric code, that identifies the specific alleles corresponding to the particular variant. For example, a measurement outcome such as the string “CC” identifies a first variant of the rs762551 SNP in which an individual has a cytosine (C) at the rs762551 position in each copy of their genetic material. A measurement outcome such as the string “AC” identifies a second variant of the rs762551 SNP in which an individual has a C in one copy and an adenine (A) in the other at the rs762551 position. A measurement outcome such as the string “AA” identifies a second variant of the rs762551 SNP in which an individual has an A at the rs762551 position in each copy of their genetic material.

A qualifier is an identifier, such as an alphanumeric code, that identifies a classification of a variant, wherein the classification may be based on the prevalence of the variant within a population, a health-related trait associated with the variant, and/or other relevant classification bases.

Qualifiers may be words or short phrases that characterize the variant. For example, “adapt” may be used to characterize variants that are uncommon and/or disadvantageous; “normal” may be used to characterize variants that are common and/or neither advantageous nor disadvantage; and “gifted” may be used to characterize variants that are uncommon and/or advantageous. Additional information may also be included within a SNP object to describe a particular variant.

In certain embodiments, measurement outcomes and qualifiers that identify and classify, respectively, the same variant are associated with each other to form a variant object associated with the SNP object. For example, variant object 252a comprises measurement outcome 260, qualifier 262. Variant object 252a is also comprises additional information 264. Additional information 264 comprises a description of the variant. For example, the additional information comprises a description of the specific health-related phenotype that an individual with the variant represented by variant object 252a exhibits or an explanation of the prevalence of the variant. A SNP object may be associated with a variant object to represent each variant of the particular SNP to which it corresponds. For example, SNP object is associated with three variant objects 252a-c.

In certain embodiments, the data structures described herein above are stored as a generic hierarchy for use in generating an individual's personal genetic profile assessment. A collection of data structures corresponding to genes, SNPs, and variants may be organized into one or more categories within a product (as visualized in FIG. 2, for example). Products can be personalized to a particular individual in order to provide them with specific information about their particular genome by populating or associating the generic product with the individual's genotyping data. In certain embodiments, a personal genetic profile assessment is used to populate an assessment graphical user interface (“assessment GUI”) through which an individual views an assessment of his/her genetic profile. In this way, the individual can view an assessment GUI that visualizes his/her personal genetic profile assessment by showing the individual the particular variants of SNPs that the individual has (e.g., organized in a hierarchy of products and categories).

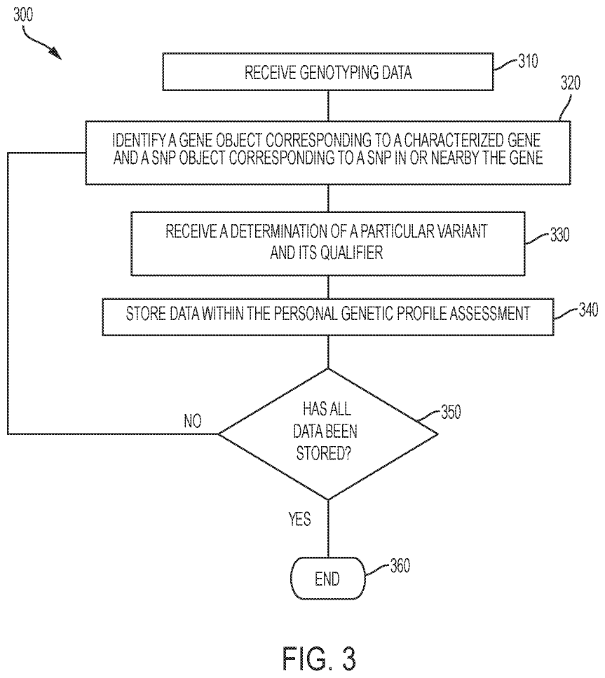

In certain embodiments, in order to populate an assessment GUI to provide to an individual, genotyping data must be added or associated to the individual's personal genetic profile assessment. FIG. 3 is a block diagram of exemplary method 300 for adding genotyping data to an individual's personal genetic profile assessment. In step 310, a processor of a computing device receives genotyping data. In step 320, the processor identifies a gene object corresponding to a gene measured in the genotyping data and a SNP object corresponding to a SNP in or nearby the gene (e.g. the SNP occurring within the gene or occurring nearby the gene (e.g. within a promotor region that influences transcription of the gene, e.g. within 5 kb upstream or downstream of the gene, e.g. within 100 kb upstream or downstream of the gene, e.g. within 500 kb upstream or downstream of the gene, e.g. within 1 Mb upstream or downstream of the gene). In certain embodiments, genotyping data is stored as a table of data in a text file where each row corresponds to a unique SNP. In step 330, a particular variant of the identified SNP object and its associated qualifier are determined based on data from genotyping measurements. For example, data corresponding to the measurement outcome of a particular variant may be stored as one or more columns at the end of each row. In step 340, the data is stored in the individual's personal genetic profile assessment. In accordance with method 300, at step 340, the data may be stored in a (previously generic) hierarchy of data structures or the data may be stored separately along with an association between the data and the identified gene object and SNP object. In any case, the stored data (and any generated and stored associations) define the personal genetic profile assessment for the individual. In step 350, the processor determines if all data of the genotyping data has been stored. If all data has not been stored in the individual's personal genetic profile assessment, then the method returns to step 320. If all data has been stored, then the method ends 360. In some embodiments, the processor determines if unstored data exists by determining if there is a row of data in the genotyping data below the just processed row.

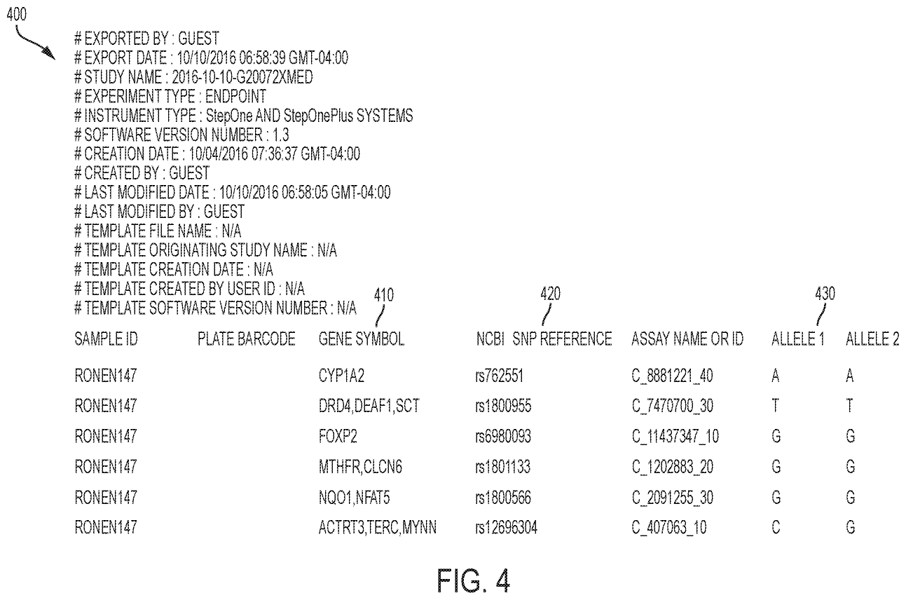

FIG. 4 shows exemplary genotyping data 400 that may be added to an individual's personal genetic profile assessment in accordance with method 300. Genotyping data may take the form of a text file saved by a user, wherein the text file is generated manually or as output from equipment for performing genotyping measurements (e.g. TaqMan™ SNP genotyping assays). FIG. 4 comprises 6 rows of genotyping data from a single biological sample (“RONEN147”). Each row corresponds to data for a different SNP. Each SNP of genotyping data 400 is identified by at least a gene identifier 410 and a SNP reference 420. The gene identifier identifies the gene with which the SNP is associated. In certain embodiments, multiple (e.g. two or more) genes are associated with the SNP (e.g. the SNP may occur nearby two or more genes and influence phenotypes associated with each of the associated genes), and, accordingly, two or more corresponding gene identifiers are listed. Each SNP in the genotyping data has a corresponding variant identified by the allele measurements 430. The measurements “allele 1” and “allele 2” for a given SNP may be compared with measurement outcomes associated with the variants of a SNP object corresponding to the given SNP to populate an individual's personal genetic profile assessment.

The genotyping data in FIG. 4 used to populate an individual's personal genetic profile assessment is generated from one or more biological samples of the individual. However, the one or more biological samples used in populating an individual's personal genetic profile assessment may also be taken from a different human or a non-human animal. In some embodiments, genotyping data is generated from one or more biological samples of a non-human animal. For example, an individual may supply biological samples of his or her pet in order to understand information about the pet's phenotype in order to assist in providing better care. The animal may be a pet or may be an animal cared for by an individual. For example, the individual may be a veterinarian or a caretaker at a zoo charged with caring for the animal. In some embodiments, genotyping data is generated from one or more biological samples of a ward to whom the individual is a guardian. For example, a parent may supply one or more biological samples to genotyping data for their child in order to improve his/her childrearing.

### Purchase Recommendations Based on Genotyping Data

Based on the various different SNP variants an individual has (and/or other genotyping data), certain supplements or combinations thereof may be useful for that individual. For example, if an individual has a particular variant of a SNP that causes him or her to be prone to weight gain (e.g., a particular variant of a SNP of the ADIPOQ gene) then it would be valuable for that individual to take supplements that help to manage or prevent weight gain and obesity. For example, if an individual has a particular variant of a SNP that causes him or her to have a reduced ability to convert beta carotene to retinol, that individual may benefit from taking a vitamin A supplement. Similarly, depending on whether an individual has particular SNP variants that influence longevity, joint health, muscle recovery, endurance and lean body mass, and skin health, different supplements may be identified that would benefit the individual.

Based on genotyping measurement results stored in an individual's personal genetic profile assessment, various relevant supplements that are of particular benefit to the individual can be identified. In certain embodiments, the identified supplements can be provided (e.g. displayed) to the individual using the same GUI that is used by the individual to view their personal genetic profile assessment. In certain embodiments, a different GUI is used by the individual to view the identified supplements.

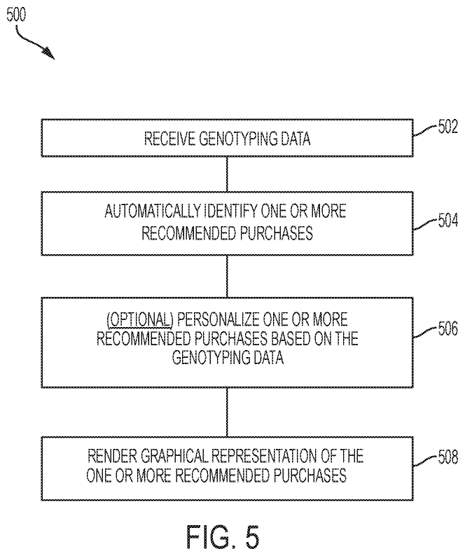

Referring now to FIG. 5, method 500 is an exemplary method for presenting a user with recommended purchases based on the particular health-related phenotypes of the user. In step 502, genotyping data of the user is received (e.g., by a processor of a computing device). In step 504, one or more recommended purchases are automatically identified based on the genotyping data. In certain embodiments, recommended purchase objects (i.e., data structures corresponding to recommended purchases) are created by a developer and associated with data structures (e.g., gene objects, SNP objects, and/or variant objects) in a user's personal genetic profile assessment based on the relevance of the corresponding recommended purchases to the corresponding genomic constituents (e.g., genes, SNPs, and/or variants). Thus, automatic identification may comprise calling or identifying one or more of those stored associations. In some embodiments, recommended purchases are identified by searching a database of all possible recommended purchases using a query comprising data from a user's personal genetic profile assessment.

For example, purchase recommendation objects that represent specific potential recommended purchases may be stored in a purchase recommendation database. Each purchase recommendation object stored in the purchase recommendation database is associated with one or more stored variant objects. The stored variant objects associated with a particular purchase recommendation object represent the particular variants of various SNPs for which the potential recommended purchase represented by the particular purchase recommendation object is recommended. For example, a purchase recommendation object representing a Vitamin A supplement could be associated with a stored variant object that represents a particular variant of a SNP that causes an individual to have a reduced ability to convert beta carotene to retinol.

A user's genotyping data (e.g., as stored in their personal genetic profile assessment) can then be used to query the purchase recommendation database to identify particular recommended purchases that will be beneficial to them. In particular, the user's genotyping data represents results of genotyping measurements performed on a biological sample from the user in order to determine the specific variants of various SNPs that are present in their genome. These results can be represented in the genotyping data via a plurality of user-specific variant objects, each of which represents the specific variant of a specific SNP that the user has in their genome.

Accordingly, the user-specific variant objects can be matched to the stored variant objects. Variant objects may be matched based on measurement outcomes and/or qualifiers that they are associated with. The purchase recommendation objects that are associated with the stored variant objects that match the user specific variant objects of the genotyping data can thus be identified to determine a set of potential recommended purchases. One or more recommended purchases can then be selected from the determined set of potential recommended purchases. In certain embodiments, all the potential recommended purchases may be selected. In certain embodiments, additional criteria, such as a user rating, cost, availability to the user, whether a particular recommended purchase conflicts with others, may be used to select the one or more recommended purchases from the determined set of one or more potential recommended purchases.

In step 508, the identified one or more recommended purchases are rendered for graphical representation (e.g., display) to the user. The graphical representation may include one or more icons and/or text for display within an assessment GUI or it may include a link (e.g., a button, hyperlink, selectable icon) that a user selects to access a separate GUI for viewing the purchase recommendations. In certain embodiments, a user may purchase purchase recommendations directly using an assessment GUI.

For example, in certain embodiments, as described above, a particular purchase recommendation is identified based on its association with a particular variant of a particular SNP. Accordingly, an icon and/or text for display that corresponds to the particular purchase recommendation may be displayed, within the assessment GUI, in a manner that visually associates it with displayed portion of the genotyping data results based on which it was identified.

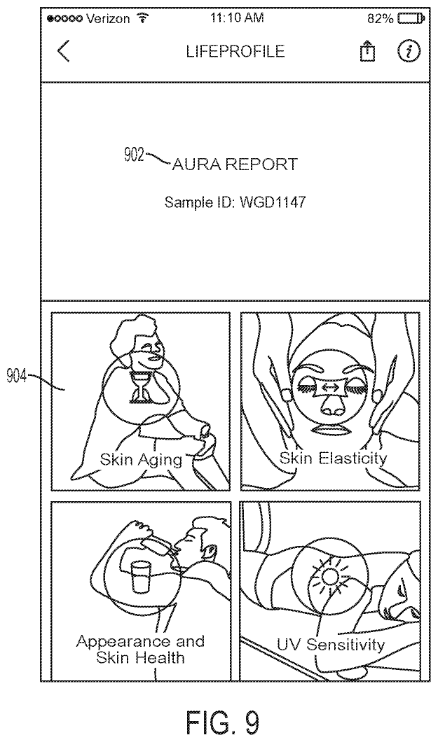

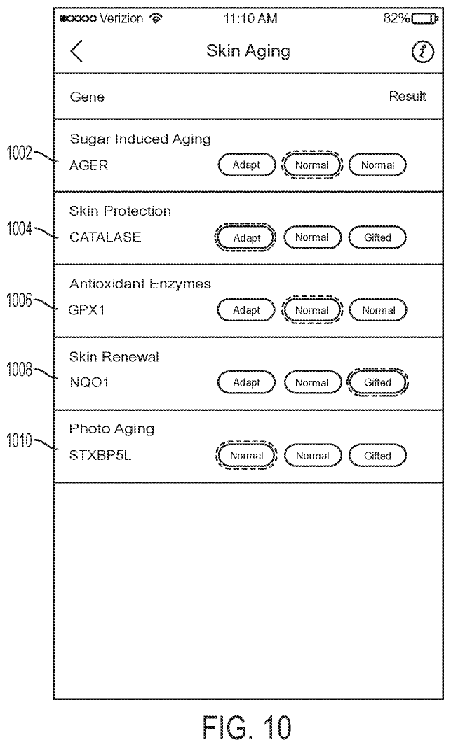

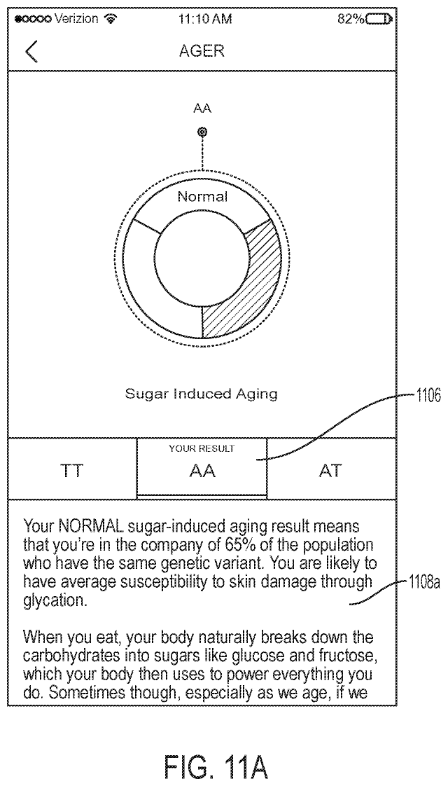

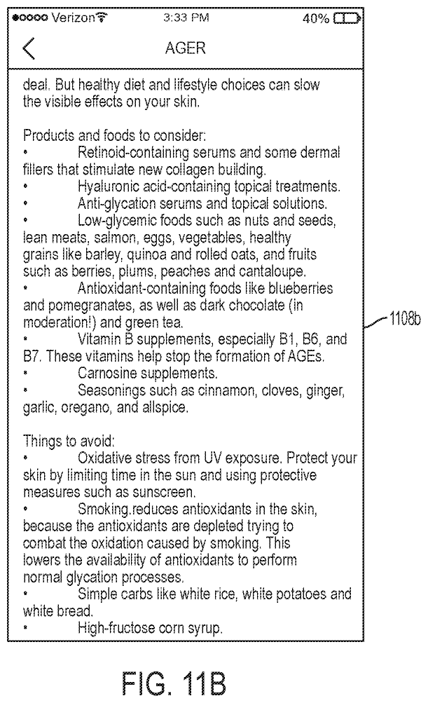

For example, in order to show a user the results of their genotyping measurements, a view of the assessment GUI may include graphics and/or text that identify the particular SNP and/or a gene with which it is associated (e.g., a name of the gene, and/or a short description of it). The view of the assessment GUI may also include graphics and/or text that identify the particular variant of the particular SNP that the user has and/or a qualifier determined based on the variant. Such graphics and/or text accordingly serve to convey to the user the particular variant of the particular SNP that they have in their genome, and any important implications for their health. Examples of assessment GUI views are shown in FIG. 9, FIG. 10, FIG. 11A, and FIG. 11B.

A particular recommended purchase identified based on its association with a particular SNP may, accordingly, be displayed in a visually associated manner to the graphics and/or text that relate to the particular SNP. For example, the icon and/or text corresponding to the particular recommended purchase may be displayed in close proximity to the graphics and/or text related to the particular SNP.

In certain embodiments, purchase of a particular recommended purchase by the user is facilitated by rendering a selectable button corresponding to the particular recommended purchase and associating the selectable with a link (e.g., a weblink) to a predefined website of a specific merchant. In this manner, a user selection of the selectable button initiates their purchase of the particular recommended purchase to which it corresponds. In certain embodiments, the user may store sets of their information that can be provided to the merchant site automatically. For example, they may store address and payment information (e.g., credit card information) in a secure database. Upon their selection of the selectable button for purchasing the particular recommended purchase, the systems and methods described herein access the user information and automatically provide it to the merchant site. In certain embodiments, all information necessary for the purchase is stored and automatically provided to the merchant site, such that the user purchase can be completed with a single click of the selectable button (e.g., no further user interaction is required).

In optional step 506, one or more recommended purchases are personalized based on the genotyping. In certain embodiments, at least some of the recommended purchases offered to a user are programs and/or therapies that are personalizable (e.g., personalized) to the user. Such recommended purchases may be personalized based on the genotyping data received in step 502. For example, a fitness program recommended to a user based generally on genotype(s) of the user may further be personalized to the user based on one or more particular genotypes. More specifically, again for example, a fitness program may be recommended based on several traits of a user, but certain particular exercises in the fitness program may be substituted based on the particular phenotype of the user that make the user more susceptible to experiencing joint inflammation and/or pain. As an additional example, a meal program recommended to a user based on health-related phenotypes that suggest the user has sugar sensitivity may be modified to exclude dairy products from the program based on lactose intolerance of the user, as determined from genotyping data.

In certain embodiments, custom meal programs may be determined for a user using a dietary profile created based on their genotyping data. The dietary profile for the user represents guidelines and/or taste preferences for the user and comprises a set of user specific dietary tags (e.g., alphanumeric strings) that identify common diets and/or allergens. For example, dietary tags such “vegetarian”, “vegan”, “pescatarian”, “low-cholesterol”, “dairy-free”, “lactose-free”, “gluten-free”, “paleo”, “low-sugar”, and the like may be used to identify various diets that, based on the user genotyping data, are recommended. For example, dietary tags such as “dairy”, “peanut”, “nut”, “gluten”, and the like, may be used to identify allergens that the user's genotyping data results indicates that they are allergic to. The dietary tags may be determined from the user genotyping data based on their association with particular variants of various different SNPs and/or qualifiers that classify them.

For example, SNPs associated with the FADS1, KCTDIO and PPARg influence cholesterol and fat storage levels. Accordingly, based on the presence of a variant and/or qualifier for any SNPs associated with these genes in a user's genotyping data, tags such as “low-cholesterol” may be added to a determined dietary profile for the user. Various dietary tags and associations between them and variant objects and/or qualifiers that identify and/or classify, respectively, specific possible variants of various SNPs may be stored, such that a dietary profile may be populated with dietary tags via automated matching between (i) user-specific variant objects and/or user-specific qualifiers from the genotyping data and (ii) stored variant objects and/or stored qualifiers.

Once determined, the user dietary profile can be used identify meal programs and specific recipes that are recommended for the user. For example, in certain embodiments, a meal database comprising a plurality of predefined meal programs, each associated with one or more program-specific dietary tags. User-specific dietary tags of the user's dietary profile can be matched to the program-specific dietary tags to identify meal programs stored in the meal database that are recommended for the user. The identified meal programs may comprise multiple recipes that the user can select from to follow a diet that will benefit their health.

In certain embodiments, the meal database comprises a plurality of recipes, each of with is associated with one or more recipe-specific dietary tags. User-specific dietary tags of the user's dietary profile can be matched to the recipe-specific dietary tags to identify recipes stored in the meal database that are recommended for the user. One or more of the recommended recipes can be selected and combined, automatically, to create a custom meal plan for the user.

In certain embodiments, the meal database comprises ingredient lists for various recipes that can be queried. Based on the ingredient list of a particular recipe, the systems and methods described herein may determine whether or not the particular recipe conforms to one or more of the diets identified by the user-specific dietary tags and/or does not comprise any allergens identified by the one or more user-specific dietary tags. This approach of querying ingredient lists of recipes may be used in place of, or in combination with querying recipe-specific dietary tags.

In certain embodiments, the custom meal plan includes information about the various recipes it comprises, such as titles of the recipes, and pictures of them. In certain embodiments, titles of the recipes and/or their pictures are graphically rendered. In certain embodiments, the custom meal plan comprises an identification of a website to which a user can subscribe to obtain ingredient lists and/or cooking procedures for one or more of the recipes it comprises. In certain embodiments, graphics and/or text corresponding to ingredient lists and/or cooking procedures for one or more recipes are graphically rendered for presentation to the user.

In certain embodiments, the custom meal plan comprises an identification of one or more specific restaurants and/or food delivery services through which the user can obtain at least one recipe of the recommended recipes (e.g., participating restaurants and/or participating food delivery services that provide recipe information for storage in the meal database).

In certain embodiments, a custom fitness program is identified and recommended to the user. In certain embodiments, the custom fitness program comprises one or more recommended workout classes (e.g., offered in the user's area; e.g., offered by participating merchants (e.g., gyms)) that are identified as recommended for the user based on their genotyping data. Identifications of workout classes may be stored in a workout class database. Each workout class may be associated with one or more variant objects and/or qualifiers that represent and/or classify, respectively, specific variants of specific SNPs. User-specific variant objects and/or qualifiers in their genotyping data can be matched to the stored variant objects and/or qualifiers to identify relevant workout classes. For example, SNPs associated with the COL5a1 gene influence joint strength and flexibility. Certain variants of SNPS associated with the COL5a1 gene render an individual prone to reduced flexibility, hypertension, and risk of injury during specific types of exercise. Accordingly, certain workout classes that, for example, offer low impact stretching and flexibility exercises may be associated with variant objects and/or qualifiers that correspond to these variants, such that they can be recommended to users that will benefit from them.

In certain embodiments, a physical fitness profile, similar to the above described dietary profile, may be determined for the user based on their genotyping data. The physical fitness profile may comprise a set of user-specific fitness tags that identify specific workout classifications (e.g., that are recommended for the user; e.g., that the user should avoid)(e.g., alphanumeric strings such as “HIIT”, “aerobic”; “cardio”; “high intensity”, “flexibility”, and the like) having been determined, by the processor, as associated with (e.g., beneficial to) the user based on their genotyping data. The user-specific fitness tags can then be used to query a workout class database comprising a plurality of workout classes, each associated with one or more program-specific fitness tags. By matching the user-specific fitness tags to program-specific fitness tags, relevant workout classes can be identified via their associate to matched program-specific fitness tags.

Once identified, the one or more recommended workout classes may be provided for presentation to the user. In certain embodiments, graphics and/or text corresponding to a recommended workout class are graphically rendered for presentation to the user. In certain embodiments, graphics and/or text representing additional information associated with the recommended workout class (e.g., one or more times when the class is offered; e.g., one or more locations (e.g., of specific gyms) at which the class is offered; e.g., a cost of the class; e.g., a link to sign up for the class) are graphically rendered for presentation to the user.

In certain embodiments, locations of gyms near the user that offer a recommended workout class are identified, for example based on location data (e.g., GPS coordinates) of the user, provided e.g., by their mobile computing device (e.g., a cell phone; e.g., a smartwatch). In certain embodiments, the location data for the user is used in combination with the identified locations of gyms offering the recommended workout class to provide a map that shows the location of the nearby gym, directions to the nearby gym, and the like, to the user. For example, lists of nearby gyms, maps, directions, and the like can be displayed on the user's mobile computing device.

Where the personal genetic profile is based on SNP variants associated with identified traits, one or a combination of products may be automatically recommended according to one or more identified traits (e.g., via reference to a look-up table or other mapping). The following are example genetic traits (e.g., informed by associated, identified SNP variants determined from a biological sample of a user) that can be part of a personal genetic profile. For the example genetic traits identified below, the corresponding genes are listed in Table 1 below, as described in Example 1. SNP variants associated with the genes listed in Table 1, accordingly, influence the genetic traits described (e.g., by influencing expression of the gene associated with the genetic trait).

### Weight Management

Genetic traits associated with weight management that can be identified based (e.g., at least in part) on SNP variants include, for example, weight regain, food reward, feeling full, appetite, obesity, hunger, sweet tooth, fatty acid sensitivity, age related metabolism, lipid metabolism, fat processing ability, feeling full, mono-unsaturated fat, and sugar sensitivity. In certain embodiments, based on a user's personal genetic profile results with respect to one or more of these traits, the system automatically identifies one or more of the following supplements which may be presented to the user as an optional in-app purchase (e.g., customized supplement packs): garcinia cambogia, CLA, raspberry ketones, green tea extract, green coffee bean extract, carbohydrate and fat blockers, tonalin, hoodia, and/or meal replacements.

### Daily Support

Genetic traits associated with an individual's need for vitamins and/or the individual's ability to effectively utilize vitamins, which can be identified based (e.g., at least in part) on SNP variants, include, for example, those involving beta carotene (vitamin A), vitamin B12, vitamin D, folate levels, vitamin B6, vitamin E, and vitamin C. In certain embodiments, based on a user's personal genetic profile results with respect to one or more of these traits, the system automatically identifies one or more of the following supplements which may be presented to the user as an optional in-app purchase (e.g., customized supplement packs): multivitamins, B complex, folate and Sam-E, vitamin A, vitamin C, vitamin D, and/or vitamin E.

### Longevity

Genetic traits associated with longevity may be identified, for example, based on SNP variants of an individual. In certain embodiments, based on a user's personal genetic profile results, the system automatically identifies one or more of the following supplements which may be presented to the user as an optional in-app purchase: oxaloacetate, curcumin, turmeric, rhodiola, carnitine, and/or N-acetylcysteine.

### Joint Health and Exercise Recovery

Genetic traits associated with an individual's joint health and ability to recover from exercise include, for example, joint strength and flexibility, joint health and injury, muscle force, muscle power, cardiorespiratory capacity, exercise recovery, strength building, and blood flow regulation. In certain embodiments, based on a user's personal genetic profile results with respect to one or more of these traits, the system automatically identifies one or more of the following supplements, which may be presented to the user as an optional in-app purchase: joint health supplements (glucosamine chondroitin, fish oil, MSM, and/or collagen), and/or muscle recovery supplements (branch chain amino acids (BCAA), glutamine, and/or whey protein powder).

### Endurance and Lean Body Mass

Genetic traits associated with an individual's endurance and lean body mass include, for example, cardiac output, oxygen capacity, VO₂ max, muscle function, energy output, muscle efficiency, cardiorespiratory capacity, blood flow regulation, lean body mass, and muscle mass. In certain embodiments, based on a user's personal genetic profile results with respect to one or more of these traits, the system automatically identifies one or more of the following supplements, which may be presented to the user as an optional in-app purchase: creatine, caffeine, beta-alanine, sodium phosphate, NO₂ (arginine), and/or pre-workout supplements.

### Skin Health

Genetic traits associated with an individual's skin health include, for example, sun sensitivity, skin protection, skin renewal, skin tone, skin protection, skin health, photo aging, and skin hydration. In certain embodiments, based on a user's personal genetic profile results with respect to one or more of these traits, the system automatically identifies one or more of the following supplements, which may be presented to the user as an optional in-app purchase: biotin, vitamin E, fern extract (sun protection), primrose, black currant oil, collagen, and/or phytoceramides.

For any of the above examples, in certain embodiments, a particular formulation of a recommended supplement may also be automatically identified and presented to a user based on the user's personal genetic profile results.

In certain embodiments, the system automatically identifies one or more recommended meal programs (e.g., via food delivery service) for rendering and presentation to a user based on the user's personal genetic profile results.

In certain embodiments, the system automatically identifies one or more recommended fitness programs, brain wave feedback programs (e.g., for stress relief), and/or behavioral programs (e.g., focus programs, ADHD assistance, improved mental acuity programs, MCI prevention programs, and/or Alzheimer's prevention programs) for rendering and presentation to a user based on the user's personal genetic profile results.

### Linking Purchase Recommendations to Personal Genetic Profile Products

In certain embodiments, purchase recommendations for supplements are determined and presented to a user from a set of stored purchase recommendations input by a developer using a purchase recommendation creation back end (e.g., a creation graphical user interface). A developer may manually or automatically upload a set of purchase recommendation objects (i.e., data structures that correspond to purchase recommendations) in order for those purchase recommendations to be available to be made to a user. For example, a developer may upload a set of purchase recommendations for supplements for a range of variants of SNPs corresponding to weight management. When users view their personal genetic profile assessment that includes, in this example, a weight management personal genetic profile product, they may then see the purchase recommendations from the set uploaded by the developer that correspond to the particular variants they have.

In some embodiments, the set is indexed and stored such that it may be queried based on a user's personal genetic profile assessment. In some embodiments, additional information associated with an object in a personal genetic profile product comprises a purchase recommendation, for a plurality of objects (e.g., wherein the purchase recommendation object defines a selectable link). In certain embodiments, purchase recommendation objects are associated with generic data structure hierarchies such that when a user's personal genetic profile assessment is formed from the user's genotyping data and a generic data structure hierarchy, the relevant associated purchase recommendations are automatically (indirectly) associated with the user's personal genetic profile assessment.

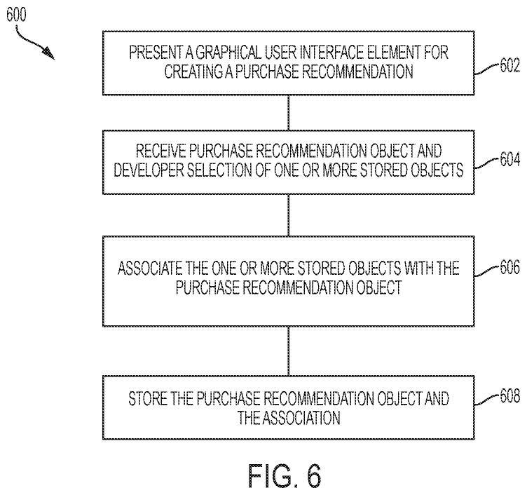

In certain embodiments, a developer creates new purchase recommendation objects and associates them with existing objects in a personal genetic profile product using a graphical user interface. Referring now to FIG. 6, method 600 is an exemplary method for creating purchase recommendation objects associated with stored objects in a personal genetic profile product. In step 602, a developer is presented with a graphical user interface element for creating a purchase recommendation object. The developer inputs data into the graphical user interface element to be included in the purchase recommendation object. Part of the graphical user interface element allows a developer to select one or more stored genomic objects to be associated with the purchase recommendation object being created. A stored genomic object is any data structure in a hierarchy of data structures that defines a personal genetic profile product (e.g., as described above). In step 604, a processor of a computing device receives a purchase recommendation object containing the data input by the developer as well as the selection of one or more stored genomic objects made by the developer. In step 606, the processor associates the one or more stored genomic objects with the purchase recommendation object. In step 608, the processor stores the purchase recommendation object and the association for further updating or retrieval (e.g., in order to populate an assessment GUI with purchase recommendations).

A graphical user interface element provided to a developer for creating a purchase recommendation object comprises one or more graphical control elements used to input data related to the purchase recommendation corresponding to the purchase recommendation object. For example, graphical control elements may be provided for entering a name or title of the purchase recommendation, descriptive text and information, a hyperlink (if the purchase recommendation is provided to users on a separate web interface or GUI), and icons used in displaying the purchase recommendation to a user. In certain embodiments, a graphical user interface element provides one or more graphical control elements (e.g., drop down lists) for a developer to select a previously created purchase recommendation object and associate it with a stored genomic object (e.g., for updating purchase recommendations for certain genotypes or health-related phenotypes based on new research or guidelines).

A purchase recommendation object may be associated with any stored object of a personal genetic profile product. In certain embodiments, purchase recommendation objects are most frequently associated with variant objects, because certain purchase recommendations are suitable only for users with a particular variant of a SNP. For example, a user with a neutral variant for a SNP corresponding to joint pain would not experience elevated joint pain or an increased likelihood of joint pain. Hence, associating a purchase recommendation object for an anti-inflammation supplement with this joint pain SNP object would lead this particular user to receive an unnecessary purchase recommendation for the anti-inflammation supplement. In contrast, a user with an “adapt” qualified variant (e.g., having a higher susceptibility to joint pain or elevated joint pain) would benefit from such a supplement recommendation. In certain embodiments, a purchase recommendation object is associated with a SNP object, gene object, category, or product if the supplement of the purchase recommendation is believed to be beneficial to all or most users regardless of the particular variant any of the users has.

In certain embodiments, purchase recommendation objects can comprise data input from a developer that causes a purchase recommendation normally shown to users with a variant of a SNP to be hidden from view of a user if the user has a particular variant of another SNP. A user may, absent all other genotyping data, receive a purchase recommendation based on a particular health-related trait they possess. However, due to a different health-related trait, the user may not receive that same purchase recommendation as the supplement being recommended would confer or increase the likelihood of conferring a negative effect based on the different health-related trait. For example, if a user's phenotype makes the user easily build muscle mass, but the user's phenotype also makes the user sensitive to sugar, based on data input by the developer, a purchase recommendation for a muscle-mass-building supplement normally provided may not be shown to the user since the supplement is high in sugar and the user has a sugar sensitivity.

Interaction with Mobile Health Devices

In certain embodiments, the systems and methods described herein provide for interaction with one or more mobile health devices of the user. Mobile health devices can be used to record health data about a user. Data recorded via a mobile health device (e.g., mobile health data) includes a range of biological/physical measurements of the user, such as their weight, glucose levels, brain activity (e.g. as measured via an EEG), and the like, as well as data about activities the user performs, such as physical activity level and diet. Biological/physical measurements can be performed via devices such as a network connected scale, and wearable brain activity monitoring devices (e.g. wearable devices capable of recording an EEG signal). Physical activity can be measured by mobile health devices such as activity tracking devices and smartphones that allow a user to record and track data about activities such as workouts, sleep, and meals via various different apps. A given mobile health devices may record one or more biological/physical measurements and/or activity measurements. Mobile health data may be recorded in an automated fashion, and/or in connection with a user interaction with the mobile health device.

Mobile health data about a user may be received and/or accessed by the systems and methods described herein and utilized in combination with the user's genotyping data (e.g. personal genetic profile assessment) to provide and/or update purchase recommendations to the user and/or to provide feedback to the user about their activities.

For example, any of the approaches described above for identifying purchase recommendations to a user based on genotyping data may be augmented by incorporating mobile health data in addition to genotyping data in the identification process. For example, if genotyping data of a user indicates that they are prone to obesity, while their mobile health data (e.g. recorded via an activity monitor, or a smartphone) shows that they have a low physical activity level, a purchase recommendation corresponding to a fitness program may be identified.

In certain embodiments, feedback about a user's activities is provided based on mobile heath data and their genotyping data. For example, mobile health data from a fitness monitor (e.g. an activity monitor, e.g. a smartphone app that tracks workouts) may be received and/or accessed and analyzed in combination with the user's personal genetic profile assessment. Such analysis can be used to tailor feedback to the user that informs them if they should modify their activities.

For example, a user's personal genetic profile assessment can provide information about a user's susceptibility to joint injury, and mobile health data can be accessed and monitored to provide feedback to the user in order to limit their risk of joint injury. For example, the GDF5 gene influences joint health. Depending on the particular variant of the GDF5 gene that a user has, they may be susceptible to joint injury. Mobile health data, such as data recorded via an activity tracking device or a smartphone app, is accessed to determine an activity level of the user. Feedback based on the user's activity level and the variant of the GDF5 gene the user has is then provided to the user in order to allow them to modify their activity level to avoid injury. For example, for each variant of the GDF5 gene, a different threshold value for a number of steps taken in a given day is determined and, if mobile health data for the user indicates that the user has exceeded the threshold number of steps, a notification is sent to the user that informs them to reduce their activity level. In another example, feedback may be based on the user's activity level and a combination of variants that may make the user particularly susceptible to a particular kind of injury or damage. For example, a user with a variant that makes the user susceptible to joint injury who also has a variant indicative of high endurance may be especially susceptible to injury, because the user may be more likely to expose himself or herself to an injury-causing level of strain.

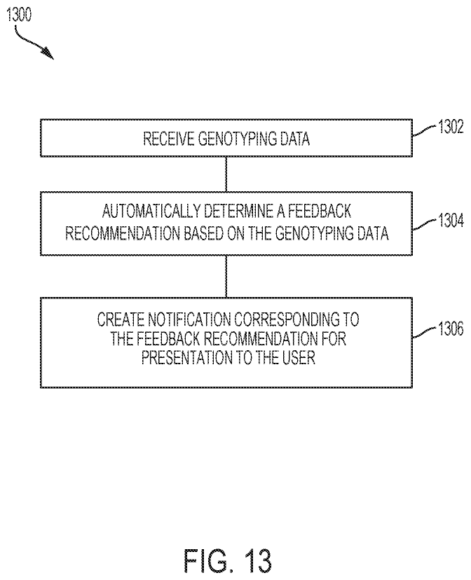

Turning to FIG. 13, in certain embodiments, feedback provided to the user takes the form of notifications that remind the user to perform one or more activities in accordance with a purchase recommendation. In the example process 1300 shown in FIG. 13, in step 1302 genotyping data for the user is received. In another step 1304, a feedback recommendation is automatically determined based on the received genotyping data. Once a feedback recommendation is determined, in another step 1306, a notification corresponding to the feedback recommendation is created for presentation to the user.

For example, various genes in an user's personal genetic profile assessment influence behavioral traits such as anxiety levels and stress response (e.g. the RGS2 gene influences anxiety and panic response). Users that have variants of these genes that make them prone to anxiety and difficulty managing stress may benefit from regular meditation. Accordingly, a purchase recommendation identified for the user based on their genotyping data may comprise a brain wave feedback program wherein the user works to manage their brain activity via monitored meditation sessions with the help of a wearable mobile health device, for example, a brain activity sensing headband. Mobile health data recorded by the wearable device may comprise a list of times and durations of meditation sessions, as well as brain wave recordings (e.g. EEG signals) during each session. Based on the number and durations of the meditation sessions an individual performs (e.g. during a given week) and the particular variants of specific behavioral genes (e.g. the RGS2 gene) that an individual has, they can be sent notifications reminding them to perform an appropriate number of meditation sessions. Mobile health data corresponding to brain wave recordings may also be monitored, and, for example, if a user appears particularly stressed (e.g. as indicated by analysis of accessed brain wave recordings), they may be instructed to perform additional meditation sessions.

In certain embodiments, feedback is provided to the user in the form of a notification. The notification may be presented to the user, for example, via a mobile health device of the user or a computing device of the user, different from the mobile health device. For example, feedback determined using mobile health data may be presented to the user directly on the mobile health device that was used to record the data, or on different device, such as a mobile computing device of the user (e.g. their smartphone). In certain embodiments, the notification that presents the feedback is a graphically rendered notification comprising one or more icons and/or alphanumeric strings. In certain embodiments, the notification comprises an auditory notification, such as an alarm or an audio message. In certain embodiments the notification comprises a haptic cue (e.g. a vibration). In certain embodiments, any combination of a graphically rendered notification, an auditory notification and a haptic cue may be provided via a user computing device.

In certain embodiments, recommended purchases identified for a user include one or more mobile health devices that are related to another recommended purchase. For example, if for a given user, a recommended purchase corresponding to a fitness program is identified, one or more mobile health devices, such as activity trackers or specific smartphone apps that facilitate the ability of the user to adhere to the fitness program are also identified. Similarly, in certain embodiments a recommended purchase corresponding to a brain wave feedback program may be linked to one or more recommended purchases corresponding to wearable brain wave monitoring and/or meditation assistance devices.

Recommended purchases may be products in the form of hardware, software, or combinations of hardware and software.

### Unlocking Genotyping Data Through Participating Merchant Rewards

In certain embodiments, particular portions of a user's genotyping data are associated with specific purchases and/or merchants not just for purposes of recommending relevant purchases, but to create promotional rewards for incentivizing user visits specific participating merchants. For example, when a user sends in a biological sample for genotyping, various SNPs may be measured, and the results stored in genotyping data for the user. A subset of the genotyping data may be labelled as unlocked, and the remainder labelled as locked, a such that the user only has access to the results of genotyping measurements of the SNPs within the unlocked subset. For example, the assessment GUI may be programmed to only display graphics conveying information about genotyping data corresponding to the unlocked subset. Graphics and/or text may also be rendered to convey to the user that portions of their genotyping data are locked (e.g., via grayed out graphics and/or text; e.g., via graphics representing a physical lock icon).

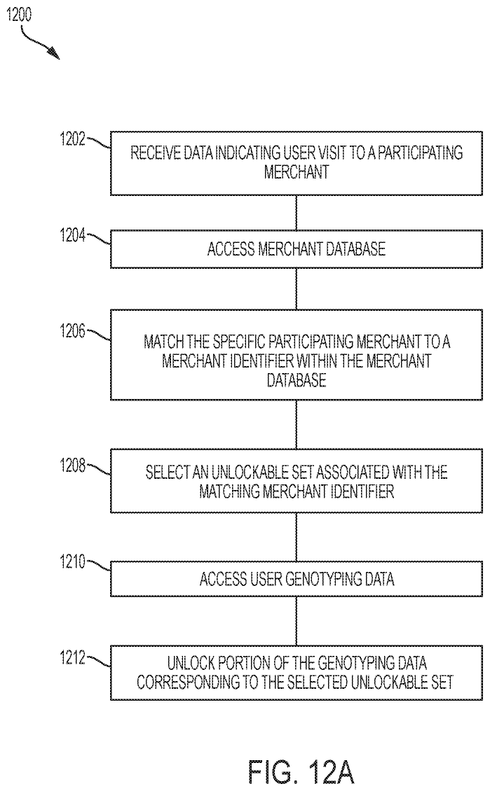

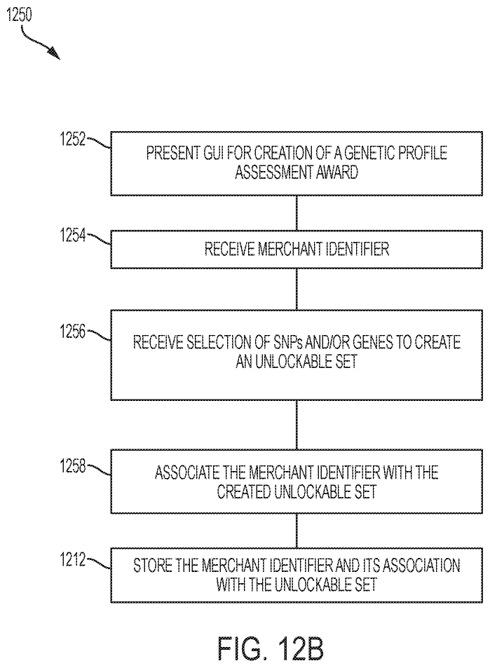

In certain embodiments, the systems and methods described herein allow a user unlock portions of their genotyping data by visiting and/or making purchases at specific participating merchants. For example, FIG. 12A shows an example process 1200 for automatically unlocking portions of a user's genotyping data based on their visiting participating merchants. In one step 1202, data indicating a user visit to a specific participating merchant is received. The data may be any of a GPS signal, an alert generated by a dedicated check-in [e.g., accomplished via scanning of a code (e.g., a Quick Response code) generated in an app on a user's mobile computing device], or an alert generated when the user begins to make a purchase at the merchant.

In another step 1204, a merchant database is accessed. The merchant database comprises a plurality of merchant identifiers, each of which identifies a particular participating merchant. Each merchant identifier is associated with one or more unlockable set(s), each of which comprises identifiers of one or more SNPs and/or genes so as to identify the specific set of genotyping data to be unlocked when the user visits a particular participating merchant. Accordingly, by matching the specific participating merchant identified in the data received at step 1202 to a merchant identifier in the merchant database, one or more unlockable set(s) to potentially be unlocked as a reward for the user visit to the specific participating merchant are identified.

Different merchants may be associated with different unlockable sets in the merchant database, such that depending on the specific participating merchant that the user visits, a different portion of their genotyping data can be unlocked. The association of different merchants with different unlockable sets may be based on what type of products the merchant sells, and their relation to different health-related phenotypes influenced by different genes and/or SNPs. For example, a merchant that sells skin care products, such as Sephora™, might wish to offer unlocking of a set of SNPs and/or genes related to skin health (e.g., part of Orig3n's AURA™ assessment) as a reward, and, accordingly, be associated with a first unlockable set comprising identifiers of SNPs and/or genes that are related to skin health. On the other hand, a merchant that sells sports equipment, such as Footlocker™, might offer unlocking of a set of SNPs and/or genes related physical fitness as a reward (e.g., part of Orig3n's FITCODE™ assessment) as a reward and, accordingly, be associated with a second unlockable set comprising identifiers of SNPs and/or genes that relate to physical fitness.

To unlock a portion of the user's genotyping data, at least one of the one or more unlockable set(s) associated with the matching merchant identifier is/are selected 1208. The genotyping data for the user is accessed 1210, and a portion of the genotyping data corresponding to the selected unlockable set that was previously labelled as locked, is modified to be labelled as unlocked 1212. Once unlocked in this manner, this portion of the user's genotyping data can be accessed and viewed by the user. In certain embodiments, a notification is sent to the user to notify them of the reward—e.g., that a portion of their genotyping data has been unlocked.

In certain embodiments, e.g., to encourage a user to visit various participating merchants, deal notifications are sent to a user to inform them of the various participating merchants, and which portions of their genotyping data can be unlocked by visiting which merchants. In certain embodiments, GPS data from the user is used to cause issuing of the deal notification is issues to the user when they are nearby a participating merchant.

In certain embodiments, unlocking of a particular unlockable set requires the user to satisfy specific requirements in addition to visiting a specific participating merchant. For example, a user may need to spend a certain amount, or purchase one or more specific promoted products. These additional requirements for unlocking a specific unlockable set can be represented by criteria stored and associated with the specific unlockable set. The data corresponding to the indication of the user visit to the specific participating merchant may include additional information about their visit (e.g., a total amount spent; e.g., a list of products purchased) that can be evaluated against the stored criteria such that upon determining that the user visit satisfies the stored criteria, the unlockable set with which it is associated can be unlocked. In certain embodiments, a notification (e.g., the deal notification) is sent to inform them of the criteria for unlocking various unlockable sets.

In certain embodiments, the systems and methods described herein allow for a developer to create such genetic profile assessment rewards that a user can unlock in the manner described above. FIG. 12B, shows an example process 1250 for creating genetic profile assessment rewards. A developer can use a GUI, presented to them, 1252 to input a merchant identifier and a selection of one or more SNPs and/or genes in order to create an unlockable set. The merchant identifier is received 1254 via the GUI, along with the selection of SNPs and/or genes 1256. An unlockable set is created from the selection of SNPs and/or genes, and associated with the received merchant identifier. The received merchant identifier and its association with the created unlockable set are stored in a merchant database, such that they can be accessed and matched in order to unlock portions of user genotyping data based on their visit to various participating merchants.

### Example 1: Personal Genetic Profile Product List

As described herein, various purchase recommendations of relevance to (e.g., beneficial for) a particular user can be identified based on results of genotyping measurements that provide information regarding their unique genetic profile. In particular, as described herein, various SNPs are associated with and may influence the expression of specific genes, which, in turn, influence various specific genetic traits. Accordingly, for a particular individual, purchase recommendations can be determined based on genotyping data that includes results of measurements of the specific variants of various different SNPs and their association with various different genes and the health related genetic traits they influence.

As described herein, for example in regard to FIG. 1 and FIG. 2, such information may be stored in an individual's personal genetic profile assessment of in the form of a flexible and intuitive hierarchical arrangement of data structures that group representations of specific SNPs and their associated genes (e.g., SNP objects and gene objects, respectively) into categories, which may, in turn, be grouped into products that represent commercial genetic test products. The representation of genetic information in this manner is also described in detail in U.S. Provisional Patent Application No. 62/436,947, filed Dec. 20, 2016, U.S. patent application Ser. No. 15/445,752, filed Feb. 28, 2017, and U.S. Provisional Patent Application No. 62/485,322, filed Apr. 13, 2017, the content of each of which are incorporated herein by reference in its entirety.

Examples of various specific genes and the specific traits that they influence are shown in Table 1. Table 1 shows examples of specific commercial genetic test products and lists the specific genes for which the commercial test measures associated SNPs to determine the particular variants that an individual has. The genes are shown along with a short description of the genetic trait that they influence (e.g., via their expression). The table also shows example groupings of sets of genes into categories, within the commercial genetic tests. An individual's personal genetic profile assessment may store their genotyping test results using the product, category, gene object, and SNP object data structures as described to represent the different commercial genotyping tests shown in Table 1, along with the particular categories, genes, and measured SNPs. For example, the commercial test AURA™ is used to represent a class of traits corresponding to skin health. The categories within this commercial test include, for example, ‘Skin Aging’, ‘Skin Elasticity’, ‘Appearance and Skin Health’, and ‘UV Sensitivity’. The category ‘Skin Aging’, for example, comprises five genetic traits (e.g., Sugar Induced Aging, Skin Protection, Antioxidant Enzymes, Skin Renewal, and Photo Aging), each of which is influenced by a specific SNP associated with a specific gene.

TABLE 1
Table 1 below shows a list of six commercial genetic test products provided by Orig3n, Inc. of Boston,
MA and the specific set of genes for which associated SNPs are measured for each commercial product.
The first level of the list outlined list provides the names of the six commercial genetic test
products - AURA ™, BLISS ™, FITCODE ™, FUEL ™, SUPERHERO ™, BLOOM ™. Genes
for which SNPs are measured are grouped into categories, shown in different rows and identified
by the lettered headings (e.g., “a) Skin Aging”, “b) Skin Elasticity”, etc.). The genes
for which different associated SNPs are measured are identified in the entries below each category.
Each gene is identified first by a short description of the genetic trait with which it is associated
(e.g., the genetic trait that is influenced by expression of the gene) (e.g., “Sugar Induced
Aging”) followed by a dash and the gene name (e.g., “AGER”). Genes listed multiple times
and identified by short descriptions that include the phrase “Part 1”, “Part 2”, etc.
are genes for which multiple associated SNPs are measured.

| 1) | AURA ™ | | |
|---|---|---|---|
| | a) | Skin Aging | |
| | | i) | Sugar Induced Aging - AGER |
| | | ii) | Skin Protection - CATALASE |
| | | iii) | Antioxidant Enzymes - GPX1 |
| | | iv) | Skin Renewal - NQO1 |
| | | v) | Photo Aging - STXBP5L |
| | b) | Skin Elasticity | |
| | | i) | Stretch Marks - FN1 |
| | | ii) | Cellulite - HIF1A |
| | | iii) | Collagen Breakdown - MMP1 |
| | | iv) | Skin Wrinkling - MMP3 |
| | c) | Appearance and Skin Health | |
| | | i) | Skin Hydration - AQP3 |
| | | ii) | Complexion - C11orf49 |
| | | iii) | Thick Hair - EDAR |
| | | iv) | Hair Graying - IRF4 |
| | | v) | Itchy Skin - OVOL1 |
| | | vi) | Monobrow - PAX3 |
| | d) | UV Sensitivity | |
| | | i) | Suntan - ASIP |
| | | ii) | Sun Sensitivity - MC1R |
| | | iii) | Skin Protection - SOD2 |
| 2) | BLISS ™ | | |
| | a) | Addiction | |
| | | i) | Nicotine Dependence - CHRNA4 |
| | | ii) | Craving - CNR1 |
| | | iii) | Marijuana Dependence - CNR1 |
| | | iv) | Heavy Drinking - GABRA2 |
| | | v) | Marijuana Dependence - PENK |
| | | vi) | Alcohol Withdrawal - SLC6A3 |
| | | vii) | Heavy Drinking - SLC6A4 |
| | b) | Feelings | |
| | | i) | Happiness - FAAH |
| | | ii) | Euphoria - OPRM1 |
| | | iii) | Empathy - OXTR |
| | | iv) | Positive Mood - SLC6A2 |
| | c) | Behavior | |
| | | i) | Caffeine Anxiety - ADORA2A |
| | | ii) | Motor Impairment - AKT1 |
| | | iii) | Paranoia Response - AKT1 |
| | | iv) | Agreeable Mood - CLOCK |
| | | v) | Food Reward - DRD2 |
| | | vi) | Risk Behavior - DRD4 |
| | | vii) | Panic - RGS2 |
| | | viii) | Compulsive - TPH2 |
| | d) | Tolerance | |
| | | i) | Warrior or Worrier - COMT |
| | | ii) | Migraine Sensitivity - ESR1 |
| | | iii) | Pain Tolerance - GCH1 |
| | | iv) | Pain Sensitivity - SCN9A |
| | | v) | Joint Pain Sensitivity - TRPV1 |
| 3) | FITCODE ™ | | |
| | a) | Endurance | |
| | | i) | Cardiac Output - ACE |
| | | ii) | Oxygen Capacity - ADRB1 |
| | | iii) | Muscle Function - ADRB2 |
| | | iv) | VO2 Max - ADRB2 |
| | | v) | Energy Output - ADRB3 |
| | | vi) | Muscle Efficiency - BDKRB2 |
| | | vii) | Aerobic Fitness - VEGF |
| | b) | Power Performance | |
| | | i) | Muscle Force - ACTN3 |
| | | ii) | Strength Building - MSTN |
| | | iii) | Blood Flow Regulation - eNOS |
| | c) | Metabolism | |
| | | i) | Fat Processing Ability - FABP2 |
| | | ii) | Feeling Full - FTO |
| | | iii) | Monounsaturated Fat - PPARg |
| | d) | Joint Health | |
| | | i) | Joint Strength and Flexibility - COL5a1 |
| | | ii) | Join Health and Injury - GDF5 |
| | e) | Exercise Recovery | |
| | | i) | Exercise Recovery - IL-6 |
| | | ii) | Cellular Health - NFE2L2 |
| | | iii) | Cell Repair - SOD2 |
| 4) | FUEL ™ | | |
| | a) | Food Sensitivity | |
| | | i) | Alcohol Tolerance - ALDH2 |
| | | ii) | Caffeine Metabolism - CYP1A2 |
| | | iii) | Lactose Intolerance - MCM6 |
| | | iv) | Cilantro Aversion - ORIOA2 |
| | | v) | Bitter Taste (Part 1) - TAS2R38 |
| | | vi) | Bitter Taste (Part 2) - TAS2R38 |
| | | vii) | Bitter Taste (Part 3) - TAS2R38 |
| | b) | Food Breakdown | |
| | | i) | Fatty Acid Response - FADS1 |
| | | ii) | Monounsaturated Fat - PPARg |
| | c) | Hunger and Weight | |
| | | i) | Weight Regain - ADIPOQ |
| | | ii) | Food Reward - DRD2 |
| | | iii) | Feeling Full - FTO |
| | | iv) | Appetite - LEPR |
| | | v) | Obesity - MC4R |
| | | vi) | Hunger - NMB |
| | | vii) | Sweet Tooth - SLC2A2 |
| | d) | Vitamins | |
| | | i) | Folate Levels - MTHFR |
| | | ii) | Vitamin B12 [symbol] FUT2 |
| | | iii) | Vitamin B6 [symbol] NBPF3 |
| | | iv) | Vitamin E [symbol] - RSU1 |
| | | v) | Vitamin C [symbol] - SLC23A1 |
| | | vi) | Beta Carotene, Vitamin A [symbol] - BCM01 |
| | | vii) | Vitamin D [symbol] - GC |
| 5) | SUPERHERO ™ | | |
| | a) | Intelligence | |
| | | i) | Learning Ability - Chromosome 6 |
| | | ii) | Language Ability - FOXP2 |
| | b) | Speed | |
| | | i) | Cardiac Output - ACE |
| | | ii) | Muscle Force - ACTN3 |
| 6) | BLOOM ™ | | |
| | a) | Enlightenment | |
| | | i) | Verbal & Numerical Reasoning/Memory [symbol] - AKAP6 |
| | | ii) | Perfect Pitch - ASAP1 |
| | | iii) | Music Pattern/Music Listening [symbol] - AVPR1A |
| | | iv) | Learning Ability [symbol] - Chromosome 6 |
| | | v) | Reading Ability [symbol] - DCDC2 |
| | | vi) | Math Ability [symbol] - FAM3A |
| | | vii) | Language Ability [symbol] - FOXP2 |
| | | viii) | Noise-induced Hearing Loss [symbol] - HSPA1L |
| | | ix) | Episodic Memory [symbol] MIR2113 |
| | | x) | Memory [symbol] - SNAP25 |
| | b) | Fitness | |
| | | i) | Cardiac Output [symbol] - ACE |
| | | ii) | Muscle Force [symbol] - ACTN3 |
| | | iii) | Joint Strength and Flexibility [symbol] - COL5a1 |
| | c) | Health | |
| | | i) | Sleep Duration [symbol] ARNTL |
| | | ii) | Adolescent Behavior [symbol] - CHRM2 |
| | | iii) | Sleep Disruption [symbol] PPARGC1B |
| | d) | Nutrition | |
| | | i) | Feeling Full [symbol] - FTO |
| | | ii) | Lactose Intolerance [symbol] - MCM6 |
| | | iii) | Cilantro Aversion [symbol] - ORIOA2 |
| | | iv) | Sweet Tooth [symbol] - SLC2A2 |

### Example 2: Viewing Purchase Recommendations within an Assessment GUI

Example 2 is an example that shows how purchase recommendations can be viewed within an assessment GUI that a user uses to view their personal genetic profile assessment on a mobile computing device.

FIG. 9 shows a screenshot of view of an assessment GUI that a user uses to view their personal genetic profile assessment. The view of the assessment GUI shown in FIG. 9 identifies a specific commercial genetic test (AURA™) via the report title 902 graphical element. The view also includes selectable icons corresponding to the different categories into which the genes and/or SNPs associated with AURA™ test are grouped (e.g., “Skin Aging”, “Skin Elasticity”, “Appearance and Skin Health”, “UV Sensitivity”). A user views their genetic test results for SNPs and/or genes that are grouped together in a particular category by selecting (e.g., via a finger press on a touch sensitive screen) the icon corresponding to the category.

As shown in this example, selection of the icon 904 corresponding to the “Skin Aging” category, brings the user to a view of the assessment GUI shown in FIG. 10. The view includes a set of selectable control elements 1002, 1004, 1006, 1008, 1010, each of which corresponds to a specific SNP measured for the user. The particular view of the assessment GUI in this example shows, for each SNP a name of a gene with which the SNP is associated along with short description of a trait influenced by, e.g., expression of, the gene. For example, a first selectable control element 1002 corresponds to a SNP associated with the AGER gene, which influences a trait described as “Sugar Induced Aging”. Likewise, a second selectable control element corresponds to a SNP associated with the CATALASE gene, which influences a trait described as “Skin Protection”.

A user can select (e.g., via a finger press on a touchscreen) any of the selectable control elements to bring up a view of the assessment GUI such as that shown in FIG. 11. The specific view shown in FIG. 11 is associated with the first selectable control element 1002, and is brought up when a user selects the first selectable control element. The view shown in FIG. 11 provides the user with detailed information about their the particular variant of the SNP associated with the AGER gene, which influences “Sugar Induced Aging”, that they have. The view includes graphical indicators representing the measurement outcomes for three possible variants of measured SNP associated with the AGER gene (e.g., “TT”, “AA”, “AT”) and indicates the particular variant that the individual has (e.g., by indicating their measurement outcome) via the blue highlighted indicator with the heading “Your Result” 1106.

A description window 1108a of the GUI shows a portion of a description associated with the specific variant that the individual has is shown at the bottom of the view. The user may scroll to view the full description. As shown in FIG. 11B, the description window 1108b may expand to occupy a majority of the screen.

Different descriptions may be associated with different variants of different SNPs that are associated with different genes. The various descriptions may be stored as a series of text files in a database and used to populate the assessment GUI. Accordingly, a user may view different descriptions relevant to the particular measurement outcome for the particular SNP that they are viewing. As shown in the portion of the description shown in FIG. 11B, various purchase recommendations may be included in the description. For example, as shown in FIG. 11B, the description may include a list of products and foods to consider, as well as things to avoid. Since each measurement outcome of each SNP can be associated with a different description (e.g., a different text file for each can be stored in a database), specific recommendations can be stored and displayed for different measurement outcomes for different SNPs.

An example set of descriptions that are stored in one implementation of an assessment GUI are shown in Tables 2 to 4 of below. Each description is associated with a particular measurement outcome of a particular SNP that is associated with a particular gene. As with Table 1 presented herein, Tables 2 to 4 show different commercial products and shows how the genes for each particular product are grouped together into categories.

Each of Tables 2 to 4 corresponds to a different commercial genetic test product provided by Orig3n, Inc. of Boston Mass. Each commercial genetic test product measures a specific set of SNPs. Each SNP measured is associated with a specific gene, such that each commercial genetic test product measures SNPs associated with a specific set of genes. Each table identifies the specific set of genes for which associated SNPs are measured in the genotyping test to which it corresponds. For each gene for which an associated SNP is measured, the three possible measurement outcomes for the SNP measurement are listed along with, for each specific measurement outcome, text that is stored as a description associated with the measurement outcome.

Each gene in the tables is identified by a short description of a genetic trait with which it is associated, such as “Sugar Induced Aging”, that is displayed in indented bold text. For each gene, the descriptions associated with the three different measurement outcomes for a SNP associated with the gene are shown. The genes are grouped into categories, each category identified by a bold underlined entry (e.g., “Skin Aging”).

TABLE 2
AURA ™ Example Descriptions
AURA ™
Skin Aging
Sugar Induced Aging

| | |
|---|---|
| Measurement | Your NORMAL sugar-induced aging result means that you're in the |
| outcome: AA | company of 65% of the population who have the same genetic variant. |
| | You are likely to have average susceptibility to skin damage through |
| | glycation. |
| | When you eat, your body naturally breaks down the carbohydrates into |
| | sugars like glucose and fructose, which your body then uses to power |
| | everything you do. Sometimes though, especially as we age, if we |
| | consume too many sugary or high-glycemic foods, the excess sugar sticks |
| | to the skin's collagen and elastin fibers, causing them to become very |
| | rigid. This is called glycation. |
| | The proteins in skin that are most prone and vulnerable to glycation are |
| | collagen and elastin. They are also the proteins that make your skin look |
| | full and youthful, so they're important to care for. When collagen and |
| | elastin link up with renegade sugars, they become discolored, weak, and |
| | less pliant. This is reflected on the skin's surface as wrinkles, sagging, and |
| | a dull complexion. Your results mean that if you consume relatively |
| | normal levels of sugar, the glycation process that gradually occurs as |
| | natural aging takes place isn't really a big deal. But healthy diet and |
| | lifestyle choices can slow the visible effects on your skin. |
| | Products and foods to consider: |
| | Retinoid-containing serums and some dermal fillers that stimulate |
| | new collagen building. |
| | Hyaluronic acid-containing topical treatments. |
| | Anti-glycation serums and topical solutions. |
| | Low-glycemic foods such as nuts and seeds, lean meats, salmon, |
| | eggs, vegetables, healthy grains like barley, quinoa and rolled oats, and |
| | fruits such as berries, plums, peaches and cantaloupe. |
| | Antioxidant-containing foods like blueberries and pomegranates, |
| | as well as dark chocolate (in moderation!) and green tea. |
| | Vitamin B supplements, especially B1, B6, and B7. These |
| | vitamins help stop the formation of AGEs. |
| | Carnosine supplements. |
| | Seasonings such as cinnamon, cloves, ginger, garlic, oregano, and |
| | allspice. |
| | Things to avoid: |
| | Oxidative stress from UV exposure. Protect your skin by limiting |
| | time in the sun and using protective measures such as sunscreen. |
| | Smoking reduces antioxidants in the skin, because the antioxidants |
| | are depleted trying to combat the oxidation caused by smoking. This |
| | lowers the availability of antioxidants to perform normal glycation |
| | processes. |
| | Simple carbs like white rice, white potatoes and white bread. |
| | High-fructose corn syrup. |
| | Consider consulting an in-store beauty adviser for products appropriate for |
| | your results or a licensed aesthetician or dermatologist for |
| | recommendations on any appropriate course of action. Before making any |
| | dietary changes, you may wish to speak with a registered dietitian |
| | nutritionist. |
| Measurement | Your NORMAL sugar-induced aging result means that you're in the |
| outcome: AT | company of 30% of the population who have the same genetic variant. |
| | You are likely to have average susceptibility to skin damage through |
| | glycation. |
| | When you eat, your body naturally breaks down the carbohydrates into |
| | sugars like glucose and fructose, which your body then uses to power |
| | everything you do. Sometimes though, especially as we age, if we |
| | consume too many sugary or high-glycemic foods, the excess sugar sticks |
| | to the skin's collagen and elastin fibers, causing them to become very |
| | rigid. This is called glycation. |
| | The proteins in skin that are most prone and vulnerable to glycation are |
| | collagen and elastin. They are also the proteins that make your skin look |
| | full and youthful, so they're important to care for. When collagen and |
| | elastin link up with renegade sugars, they become discolored, weak and |
| | less pliant. This is reflected on the skin's surface as wrinkles, sagging and |
| | a dull complexion. Your results mean that if you consume relatively |
| | normal levels of sugar, the glycation process that gradually occurs as |
| | natural aging takes place isn't really a big deal. But healthy diet and |
| | lifestyle choices can slow the visible effects on your skin. |
| | Products and foods to consider: |
| | Retinoid-containing serums and some dermal fillers that stimulate |
| | new collagen building. |
| | Hyaluronic acid-containing topical treatments. |
| | Anti-glycation serums and topical solutions. |
| | Low-glycemic foods such as nuts and seeds, lean meats, salmon, |
| | eggs, vegetables, healthy grains like barley, quinoa and rolled oats, and |
| | fruits such as berries, plums, peaches and cantaloupe. |
| | Antioxidant-containing foods like blueberries and pomegranates, |
| | as well as dark chocolate (in moderation!) and green tea. |
| | Vitamin B supplements, especially B1, B6, and B7. These |
| | vitamins help stop the formation of AGEs. |
| | Carnosine supplements. |
| | Seasonings such as cinnamon, cloves, ginger, garlic, oregano, and |
| | allspice. |
| | Things to avoid: |
| | Oxidative stress from UV exposure. Protect your skin by limiting |
| | time in the sun and using protective measures such as sunscreen. |
| | Smoking; it reduces antioxidants in the skin, because the |
| | antioxidants are depleted trying to combat the oxidation caused by |
| | smoking. This lowers the availability of antioxidants to perform normal |
| | glycation processes. |
| | Simple carbs like white rice, white potatoes and white bread. |
| | High-fructose corn syrup. |
| | Consider consulting an in-store beauty adviser for products appropriate for |
| | your results or a licensed aesthetician or dermatologist for |
| | recommendations on any appropriate course of action. Before making any |
| | dietary changes, you may wish to speak with a registered dietitian |
| | nutritionist. |
| Measurement | Your ADAPT result means that you are much more susceptible to the |
| outcome: TT | sugar-induced aging that results in skin damage from glycation. With this |
| | result, found in only 5% of the population, you may be prone to skin |
| | looseness, cracking, thinning or dullness. |
| | When you eat food, your body naturally breaks down the carbohydrates |
| | into sugars like glucose and fructose, which your body uses to power |
| | everything you do. Sometimes though, especially when we get older, if we |
| | consume too many sugary or high-glycemic foods, the excess sugar from |
| | your body sticks to the skin's collagen and elastin fibers, causing them to |
| | become very rigid. This is called glycation. |
| | The proteins in skin that are most prone and vulnerable to glycation are |
| | collagen and elastin. They are also the proteins that make your skin look |
| | full and youthful, so they're important to care for. When collagen and |
| | elastin link up with renegade sugars, they become discolored, weak and |
| | less pliant. This is reflected on the skin's surface as wrinkles, sagging and |
| | a dull complexion. Your result means that if you want to maintain bright, |
| | firm and healthy skin, you must be extra mindful of the foods you eat, and |
| | you may want to use products that will slow or combat the production and |
| | formation of advanced glycation end-products (AGEs). While glycation is |
| | a natural process and can't be stopped entirely, lifestyle choices can slow |
| | or alleviate the visible effects on your skin. |
| | Products and foods to consider: |
| | Retinoid-containing serums and some dermal fillers that stimulate |
| | new collagen building. |
| | Hyaluronic acid-containing topical treatments. |
| | Anti-glycation serums and topical solutions. |
| | Low-glycemic foods such as nuts and seeds, lean meats, salmon, |
| | eggs, vegetables, healthy grains like barley, quinoa and rolled oats, and |
| | fruits such as berries, plums, peaches and cantaloupe. |
| | Antioxidant-containing foods like blueberries and pomegranates, |
| | as well as dark chocolate (in moderation!) and green tea. |
| | Vitamin B supplements, especially B1, B6 and B7. These vitamins |
| | help stop the formation of AGEs. |
| | Carnosine supplements. |
| | Seasonings such as cinnamon, cloves, ginger, garlic, oregano and |
| | allspice. |
| | Actions to consider: |
| | Control your blood sugar levels. Blood sugar spikes can affect the |
| | condition of your skin, so try to fuel your body with low-glycemic foods. |
| | Eat small portions every three to four hours. |
| | Weight train. Muscles actually consume glucose, so the more |
| | muscle you have, the more glucose your body will eat up. |
| | Things to avoid or limit: |
| | Oxidative stress from UV exposure. Protect your skin by limiting |
| | time in the sun and using protective measure such as sunscreen |
| | Smoking. Smoking reduces antioxidants in the skin, because the |
| | antioxidants are depleted trying to combat the oxidation caused by |
| | smoking. This lowers the availability of antioxidants to perform normal |
| | glycation processes |
| | Blackened meat-barbequing, searing and broiling meat actually |
| | creates the harmful AGE molecules in the food, so if you eat them, you |
| | are actually adding to what is already existing or occurring in your body. |
| | In fact, the browning reaction of cooking meat is actually the same thing |
| | that happens to the collagen in our bodies, just at a much faster rate! |
| | Simple carbs like white rice, white potatoes, and white bread |
| | High fructose corn syrup |
| | Things to avoid: |
| | Oxidative stress from UV exposure. Protect your skin by limiting |
| | time in the sun and using protective measures such as sunscreen. |
| | Smoking; it reduces antioxidants in the skin, because the |
| | antioxidants are depleted trying to combat the oxidation caused by |
| | smoking. This lowers the availability of antioxidants to perform normal |
| | glycation processes. |
| | Blackened meat; barbecuing, searing and broiling meat actually |
| | creates the harmful AGE molecules in the food, so if you eat them, you |
| | are actually adding to what already exists or occurs in your body. In fact, |
| | the browning reaction of cooking meat is the same thing that happens to |
| | the collagen in our bodies-just at a much faster rate! |
| | Simple carbs like white rice, white potatoes and white bread. |
| | High-fructose corn syrup. |
| | Consider consulting an in-store beauty adviser for products appropriate for |
| | your results or a licensed aesthetician or dermatologist for |
| | recommendations on any appropriate course of action. Before making any |
| | dietary changes, you may wish to speak with a registered dietitian |
| | nutritionist. |

Antioxidant Enzymes

| | |
|---|---|
| Measurement | Your result means that you likely have NORMAL antioxidant activity |
| outcome: CC | protecting you from the effects of skin toxins. You, along with the 60% of |
| | the population who also have this genetic variant, have average |
| | susceptibility to skin damage due to environmental factors. The skin |
| | serves as the barrier between the body and the external environment and |
| | therefore is in constant contact with stressors like sunlight and pollutants, |
| | which are major contributing factors to oxidative stress. |
| | Antioxidant enzymes are the main line of defense against free radicals in |
| | our cells. Free radicals are the waste products of almost every metabolic |
| | reaction our body has, which is to say that they are created by the normal |
| | bodily functions necessary for us to live. Free radicals can cause damage |
| | to our cells if they are not neutralized by antioxidants. Normally our body |
| | keeps these free radicals under control by producing antioxidants |
| | internally; however, exposure to environmental toxins can reduce our |
| | natural supply of antioxidants and increase free radical production. |
| | We can limit the oxidative stress put on our bodies by eating and living |
| | healthily and doing at least 30 minutes of physical activity every day. We |
| | can also take antioxidants to supplement our diet. |
| | Some suggestions for maximizing antioxidant intake include: |
| | So-called “superfoods” are among the highest in antioxidants; |
| | adding them to your diet can be greatly beneficial. Some to consider: |
| | berries, pomegranate, apples, tea, green leafy vegetables, broccoli, garlic, |
| | walnuts, herbs and spices. (Other good sources of antioxidants include |
| | apricots, artichokes, carrots, kidney beans, pecans, plums and sweet |
| | potatoes.) |
| | Pair foods to boost antioxidant properties, such as tomato with |
| | olives, spinach with oranges, dark chocolate with red wine, onions with |
| | garlic. |
| | Consume antioxidant-rich foods in meals that include oil, as |
| | antioxidants are best absorbed when oil is present. |
| | Some suggestions for limiting free radical production include reducing |
| | exposure to external sources of oxidants like alcohol, cigarette |
| | smoke, stress and excessive sun; these environmental factors can generate |
| | so many free radicals that our normal antioxidant defenses become |
| | overwhelmed, leaving us vulnerable to cell damage and disease. |
| | Consider consulting an in-store beauty adviser for products appropriate for |
| | your results or a licensed aesthetician or dermatologist for |
| | recommendations on any appropriate course of action. Before making any |
| | dietary changes, you may wish to speak with a registered dietitian |
| | nutritionist. |
| Measurement | Your result means that you likely have NORMAL antioxidant activity |
| outcome: CT | protecting you from the effects of skin toxins. You, along with the 35% of |
| | the population who also have this genetic variant, have average |
| | susceptibility to skin damage due to environmental factors. The skin |
| | serves as the barrier between the body and the external environment and |
| | therefore is in constant contact with stressors like sunlight and pollutants, |
| | which are major contributing factors to oxidative stress. |
| | Antioxidant enzymes are the main line of defense against free radicals in |
| | our cells. Free radicals are the waste products of almost every metabolic |
| | reaction our body has, which is to say that they are created by the normal |
| | bodily functions necessary for us to live. Free radicals can cause damage |
| | to our cells if they are not neutralized by antioxidants. Normally our body |
| | keeps these free radicals under control by producing antioxidants |
| | internally; however, exposure to environmental toxins can reduce our |
| | natural supply of antioxidants and increase free radical production. |
| | We can limit the oxidative stress put on our bodies by eating and living |
| | healthily and doing at least 30 minutes of physical activity every day. We |
| | can also take antioxidants to supplement our diet. |
| | Some suggestions for maximizing antioxidant intake include: |
| | So-called “superfoods” are among the highest in antioxidants; |
| | adding them to your diet can be greatly beneficial. Some to consider: |
| | berries, pomegranate, apples, tea, green leafy vegetables, broccoli, garlic, |
| | walnuts, herbs, and spices. (Other good sources of antioxidants include |
| | apricots, artichokes, carrots, kidney beans, pecans, plums and sweet |
| | potatoes.) |
| | Pair foods to boost antioxidant properties, such as tomato with |
| | olives, spinach with oranges, dark chocolate with red wine, onions with |
| | garlic. |
| | Consume antioxidant-rich foods in meals that include oil, as |
| | antioxidants are best absorbed when oil is present. |
| | Some suggestions for limiting free radical production include reducing |
| | exposure to external sources of oxidants like alcohol, cigarette |
| | smoke, stress and excessive sun; these environmental factors can generate |
| | so many free radicals that our normal antioxidant defenses become |
| | overwhelmed, leaving us vulnerable to cell damage and disease. |
| | Consider consulting an in-store beauty adviser for products appropriate for |
| | your results or a licensed aesthetician or dermatologist for |
| | recommendations on any appropriate course of action. Before making any |
| | dietary changes, you may wish to speak with a registered dietitian |
| | nutritionist. |
| Measurement | Your ADAPT result is quite rare (only 5% of people have this genetic |
| outcome: TT | variant), and it means that you may be more susceptible to decreased |
| | antioxidant activity which can lead to skin damage. Antioxidants fight the |
| | free radicals that are created by oxidative stress. The skin serves as the |
| | barrier between the body and the external environment and therefore is in |
| | constant contact with stressors like sunlight and pollutants, which are |
| | major contributing factors to oxidative stress. |
| | Antioxidant enzymes are the main line of defense against free radicals in |
| | our cells. Free radicals are the waste products of almost every metabolic |
| | reaction our body has, which is to say that they are created by the normal |
| | bodily functions necessary for us to live. Free radicals can cause damage |
| | to our cells if they are not neutralized by antioxidants. Normally our body |
| | keeps these free radicals under control by producing antioxidants |
| | internally; however, exposure to environmental toxins can reduce our |
| | natural supply of antioxidants and increase free radical production. Since |
| | your genetic variant means that you produce fewer antioxidant enzymes, |
| | you need to be particularly mindful of the environmental stresses to your |
| | skin and make efforts to take preventive measures such as increasing |
| | antioxidant intake and using appropriate products. |
| | Some suggestions for maximizing antioxidant intake include: |
| | So-called “superfoods” are among the highest in antioxidants; |
| | adding them to your diet can be greatly beneficial. Some to consider: |
| | berries, pomegranate, apples, tea, green leafy vegetables, broccoli, garlic, |
| | walnuts, herbs and spices. (Other good sources of antioxidants include |
| | apricots, artichokes, carrots, kidney beans, pecans, plums and sweet |
| | potatoes.) |
| | Pair foods to boost antioxidant properties, such as tomato with |
| | olives, spinach with oranges, dark chocolate with red wine, onions with |
| | garlic. |
| | Consume antioxidant-rich foods in meals that include oil, as |
| | antioxidants are best absorbed when oil is present. |
| | Include vitamin C and vitamin E in your diet. |
| | Consider using topical products that contain antioxidants such as: |
| | Retinol |
| | Peptide blends |
| | Resveratrol |
| | Note that sunlight deactivates many antioxidants, so while antioxidants |
| | found in these products will absorb free radicals before they can harm the |
| | skin, the antioxidant is destroyed in the process and therefore is never |
| | fully absorbed into your cells (where it is really needed). Using topical |
| | products at night will increase their defensive and reparative effects. |
| | It is critical to reduce environmental exposure to oxidative damage, so |
| | limit prolonged and direct exposure to the sun, use sunscreen when you |
| | are outdoors, do not smoke, reduce exposure to pesticides, pollutants and |
| | heavy metals, and limit alcohol intake. |
| | Consider consulting an in-store beauty adviser for products appropriate for |
| | your results or a licensed aesthetician or dermatologist for |
| | recommendations on any appropriate course of action. Before making any |
| | dietary changes, you may wish to speak with a registered dietitian |
| | nutritionist. |

Skin Elasticity
Cellulite

| | |
|---|---|
| Measurement | You are a part of almost 80% of the population with the ADAPT result. |
| outcome: CC | Your genetic variant means that you are more likely to have cellulite. |
| | Cellulite is a cosmetic condition that gives skin a rippled or dimpled |
| | appearance. It is caused by the herniation (or protrusion) of subcutaneous |
| | fat within fibrous connective tissue. This means that parts of the fat layer |
| | right under the skin starts to push up. It is most commonly found in the |
| | thighs and buttocks, particularly in post-adolescent women. It tends to |
| | increase with age. With your result, you may want to consider proactive or |
| | preventive measures to improve the appearance or slow the advancement |
| | of cellulite. |
| | Cellulite can be difficult to treat as it tends to be unresponsive to |
| | therapies. However, there are many non-invasive treatment options to |
| | consider, including the following: |
| | Use retinol cream or serum, as well as creams and topical products |
| | targeted at improving cellulite. |
| | Vigorously massage the skin to break up subcutaneous fatty |
| | deposits. |
| | Make lifestyle changes to support weight loss and increased |
| | exercise. |
| | Consume gelatin, which contains the collagen that makes skin firm |
| | and elastic. |
| | Dry brush skin regularly. |
| | Coffee scrubs - the caffeine stimulates tightening of the skin, |
| | while the exfoliation properties remove dead and dull cells. |
| | Take omega-3 supplements. |
| Measurement | This GIFTED result means that you are less likely to have cellulite. While |
| outcome: CT | you have more protection with this gene variant, it never hurts to make |
| | efforts to maintain uniform texture and prevent future cellulite. |
| | Cellulite is a cosmetic condition that gives skin a rippled or dimpled |
| | appearance. It is caused by the herniation (or protrusion) of subcutaneous |
| | fat within fibrous connective tissue. This means that parts of the fat layer |
| | right under the skin starts to push up. It is most commonly found in the |
| | thighs and buttocks, particularly in post-adolescent women. It tends to |
| | increase with age. With your result, you may not need to maintain a |
| | healthy diet and regular exercise to keep your skin looking smooth and |
| | firm, but both will help you look and feel your best. |
| Measurement | This GIFTED result is rare; it means that you are lucky enough to have |
| outcome: TT | the most protective form of the gene. With this result, you are least likely |
| | to have cellulite. |
| | Cellulite is a cosmetic condition that gives skin a rippled or dimpled |
| | appearance. It is caused by the herniation (or protrusion) of subcutaneous |
| | fat within fibrous connective tissue. This means that parts of the fat layer |
| | right under the skin starts to push up. It is most commonly found in the |
| | thighs and buttocks, particularly in post-adolescent women. |
| | You will likely not need any cellulite-specific creams or treatments to help |
| | maintain uniform texture and prevent future cellulite. |
| | Consider consulting an in-store beauty adviser for products appropriate for |
| | your results or a licensed aesthetician or dermatologist for |
| | recommendations on any appropriate course of action. Before making any |
| | dietary changes, you may wish to speak with a registered dietitian |
| | nutritionist. |

Appearance and Skin Health
Skin Hydration

| | |
|---|---|
| Measurement | Your ADAPT result places you with 10% of the population who have this |
| outcome: CC | rare result. Research has shown that a person with this result has a genetic |
| | predisposition for lower skin hydration as compared to others. Low skin |
| | hydration can lead to dry, dull or itchy skin. You may need to use |
| | moisturizer regularly, especially in winter or dry areas such as deserts. |
| | You may consider using hydration masks, creams and lotions to give your |
| | skin the hydration it lacks. Drinking plenty of water, as well as beverages |
| | that include electrolytes, can help as well. |
| | Do not use (or use sparingly) such drying products as astringents or |
| | formulations containing calamine lotion, propylene glycol or witch hazel. |
| | Be cautious about using acne-reducing products that do not have added |
| | moisturizers, since they will work against your naturally dry skin. Look |
| | for facial cleansing products that are formulated for sensitive or dry skin. |
| | You may want to incorporate some of these beauty habits into your |
| | routine: |
| | Do not take excessively hot showers. Hot showers strip your skin |
| | of oils that help retain moisture. |
| | Reduce use of conventional soaps or bath gels that contain |
| | ingredients like petroleum waxes, parabens and phthalates. |
| | Apply moisturizer right after the shower to lock in and restore oils. |
| | Apply oil to moisture. It may seem counterintuitive if you are |
| | worried about oily skin, but oils moisturize better than some lotions or |
| | creams. |
| | Consider consulting an in-store beauty adviser for products appropriate for |
| | your results or a licensed aesthetician or dermatologist for |
| | recommendations on any appropriate course of action. Before making any |
| | dietary changes, you may wish to speak with a registered dietitian |
| | nutritionist. |
| Measurement | Your GIFTED result is the most common one, placing you with 50% of |
| outcome: TT | the population. Studies have shown that you have a genetic predisposition |
| | for good skin hydration, resulting in healthy skin with good elasticity. |
| | To maintain your healthy hydrated skin, consider incorporating these |
| | beauty habits into your routine: |
| | Do not take excessively hot showers. Hot showers strip your skin |
| | of oils that help retain moisture. |
| | Reduce use of conventional soaps or bath gels that contain |
| | ingredients like petroleum waxes, parabens and phthalates. |
| | Apply moisturizer right after the shower to lock in and restore oils. |
| | Apply oil to moisture. It may seem counterintuitive if you are |
| | worried about oily skin, but oils moisturize better than some lotions or |
| | creams. |
| | Consider consulting an in-store beauty adviser for products appropriate for |
| | your results or a licensed aesthetician or dermatologist for |
| | recommendations on any appropriate course of action. Before making any |
| | dietary changes, you may wish to speak with a registered dietitian |
| | nutritionist. |
| Measurement | Your NORMAL result (found in 40% of the population) means that you |
| outcome: CT | are likely to have intermediate skin hydration. You may want to increase |
| | moisturizing practices in the drier months of winter or in dry climates |
| | such as the desert. Consider using moisturizing lotion or cleansing |
| | products, and drink water or beverages containing electrolytes. |
| | Consider how your skin feels and looks before using products that might |
| | over-dry it, such as solutions containing calamine lotion, propylene glycol |
| | or witch hazel. Be cautious about using acne-reducing products on a |
| | regular basis that do not have added moisturizers. |
| | You may want to incorporate some of these beauty habits into your |
| | routine: |
| | Limit excessively hot showers. Hot showers strip your skin of oils |
| | that help retain moisture. |
| | Reduce use of conventional soaps or bath gels that contain |
| | ingredients like petroleum waxes, parabens and phthalates. |
| | Apply moisturizer right after the shower to lock in and restore oils. |
| | Consider consulting an in-store beauty adviser for products appropriate for |
| | your results or a licensed aesthetician or dermatologist for |
| | recommendations on any appropriate course of action. Before making any |
| | dietary changes, you may wish to speak with a registered dietitian |
| | nutritionist. |

TABLE 3
FITCODE ™ Example Descriptions
FITCODE ™
Endurance
Cardiac Output

| | |
|---|---|
| Measurement outcome: | With this result you are rare and an outlier. Only 15% of the population |
| GG | have GG. People with this result can have more cardiac output with |
| | increased strength, but would do so under higher cardiac strain. This |
| | means you have much less endurance but are very strong. Studies have |
| | found that people with GG may be better at exercises and activities |
| | requiring extraordinary strength for shorter periods of time. |
| | People like you will be likely to excel at exercises with rep ranges of 1-5. |
| | Activities like sprinting and high intensity interval training and |
| | workouts that get your heart rate really high for a short period of time |
| | are preferred for this result. An example would be if this person were to |
| | program a rowing workout the ideal would be to do 30 seconds as hard |
| | as they can followed by resting for a minute. People with this result are |
| | likely to be similar to power lifters. Consider working with a Certified |
| | Personal Trainer and consult your physician before undertaking any new |
| | exercise programs. |
| Measurement outcome: | This result is found in 50% of the population. This gene has been |
| AA | studied extensively and controls regulation of blood vessel constriction |
| | and therefore controls how efficient your muscles work. People with |
| | AA can have less cardiac strain under exertion, and therefore often excel |
| | at exercises requiring endurance. People with this result will gravitate |
| | towards marathons, long endurance bike races and any exercises that |
| | require steady and consistent cardiac output. People like you will be |
| | more likely to do well at 5k races rather than a 40 yard dash. |
| | People with this genetic makeup will struggle with short burst high |
| | intensity workouts but excel at moderate heart rate moderate duration |
| | workouts. Consider working with a Certified Personal Trainer and |
| | consult your physician before undertaking any new exercise programs. |
| Measurement outcome: | This gene has been studied extensively and controls regulation of blood |
| AG | vessel constriction and therefore controls how efficient your muscles |
| | work. People with AG can have less cardiac strain under exertion, and |
| | therefore often excel at exercises requiring endurance. People with this |
| | result will gravitate towards marathons, long endurance bike races and |
| | any exercises that require steady and consistent cardiac output. People |
| | like you will be more likely to do well at 5k races rather than a 40 yard |
| | dash. |
| | People with this genetic makeup will struggle with short burst high |
| | intensity workouts but excel at moderate heart rate moderate duration |
| | workouts. Consider working with a Certified Personal Trainer and |
| | consult your physician before undertaking any new exercise programs. |

Oxygen Capacity

| | |
|---|---|
| Measurement outcome: | People with this result are likely to have greater oxygen capacity and |
| CG | cardiac function and to demonstrate better endurance in long distance |
| | sports. People should do a 30 minute moderate intensity circuit rather |
| | than a 10 minute high intensity circuit. Consider working with a |
| | Certified Personal Trainer and consult your physician before undertaking |
| | any new exercise programs. |
| Measurement outcome: | This result is rare and found in 10% of the population. People with this |
| GG | result have been found to have a slower resting heart rate than those with |
| | the CC or CG results. As a result, GG is also associated with the benefit |
| | of lower heart strain under exercise. Your heart works less hard to pump |
| | blood throughout your body. |
| Measurement outcome: | This result is found in 50% of the population. This gene has a role in |
| CC | your heart rate and function. People like you with this result are likely to |
| | have greater oxygen capacity and cardiac function and can demonstrate |
| | better endurance in long distance sports such as running and triathlons. |
| | An example of weight training would be to be in an endurance range of |
| | 10-20 reps vs fewer higher weight reps. Consider working with a |
| | Certified Personal Trainer and consult your physician before undertaking |
| | any new exercise programs. |

Power Performance
Muscle Force

| | |
|---|---|
| Measurement outcome: | This genotype is quite rare and found in 10% of the population. This |
| TT | result is associated with few or no fast twitch muscles and therefore a |
| | lower capacity for absorption and transmission of muscle force during |
| | rapid contraction. |
| | Fast twitch muscle fibers are responsible for quick movements such as |
| | punch, jump or a sudden burst of energy. A lack of fast twitch muscle |
| | fibers also mean it will be more difficult to put on muscle mass quickly. |
| | People with this result may not be as inherently good at sports that |
| | require quick sudden bursts of energy compared to those who have CC |
| | and CT results. This result does not mean you should not take up |
| | activities such as boxing or sprinting rather you may just not excel at this |
| | type of activity. |
| | You may be likely to excel at more endurance types of activities such as |
| | biking, hiking, triathlons, rock climbing, cross country skiing and |
| | swimming. An ideal training program would be to consider yoga and |
| | pilates as strengthening exercises with higher reps of lower resistance or |
| | with lower weights. Consider working with a certified personal trainer |
| | and consult your physician before undertaking any new exercise |
| | programs. |
| Measurement outcome: | This genotype is found in 45% of the general population. With this |
| TC | genotype, a person has an intermediate number of fast-twitch muscles |
| | and therefore has the capacity for absorption and transmission of muscle |
| | force during rapid contraction, and the ability to promote growth of fast |
| | twitch muscle fibers, but with not as many fast twitch fibers as those |
| | with CC. |
| | Fast twitch muscle fibers are responsible for quick movements such as |
| | punch, jump or a sudden burst of energy. |
| | With this result you can take your training program into endurance or |
| | high impact sports. For an endurance targeted program you may be |
| | targeting activities such as biking, hiking, triathlons, rock climbing, |
| | cross country skiing and swimming. An ideal training program would |
| | be to consider yoga and pilates as strengthening exercises with higher |
| | reps of lower resistance or with lower weights. |
| | For a more power sports program you may be targeting boxing, |
| | basketball, sprinting, football or other high output sports. For this type |
| | of training program consider lower repetitions and higher |
| | weight/resistance exercises. Consider working with a certified personal |
| | trainer and consult your physician before undertaking any new exercise |
| | programs. |
| Measurement outcome: | This genotype is found in 45% of the general population. This gene |
| CC | makes a protein found in fast-twitch muscles and therefore contributes to |
| | your ability to perform better in sports requiring power such as sprinting. |
| | Studies have found that more elite athletes have this genotype. |
| | Fast twitch muscle fibers are responsible for quick movements such as |
| | punch, jump or a sudden burst of energy. For a power sports program |
| | you may be targeting boxing, basketball, sprinting, football or other high |
| | output sports. For this type of training program consider lower |
| | repetitions and higher weight/resistance exercises with a focus on |
| | jumping, throwing and fast movement activities such as vertical jumps |
| | and burpees. |
| | If you are gifted here and in Vo2Max as well as endurance you may be |
| | highly suited for activities like rowing which requires a combination of |
| | those three types of genes. Consider working with a certified personal |
| | trainer and consult your physician before undertaking any new exercise |
| | programs. |

Strength Building

| | |
|---|---|
| Measurement outcome: | You have a very rare result found in only 1-2% of the population. This |
| CC | gene functions in producing a muscle protein important for controlling |
| | your muscle mass. Studies have shown that people with this result tend |
| | to have greater muscle mass and therefore are able to produce peak |
| | power during muscle contraction. |
| | People with this result can put on an extraordinary amount of muscle |
| | mass with the right exercise and nutritional program. Typical people in |
| | this category would be linebackers, power lifters and bodybuilders. |
| | Workout programs would include training programs that would be |
| | targeting athletic performance over fat loss or conditioning. |
| | If you are among the 1-2% population and want to be really lean and |
| | thin you would want to focus on high intensity interval training vs. 8-10 |
| | reps of heavy compound movements. If the goal is to add mass then |
| | lower reps and strength training would be recommended. |
| | Consider working with a certified personal trainer and consult your |
| | physician before undertaking any new exercise programs. |
| Measurement outcome: | This result is most common, accounting for 90% of the population. |
| TT | Unlike people with CC or CT, people like you have a tendency to have |
| | average muscle mass and therefore average strength. People with this |
| | result can take on a training program in any direction and likely see |
| | results. |
| | Consider working with a certified personal trainer and consult your |
| | physician before undertaking any new exercise programs. |
| Measurement outcome: | You have a rare result found in only 8-10% of the population. Similar to |
| CT | people with CC, studies have shown that people with this variant tend to |
| | be able to build greater muscle mass and therefore greater peak power |
| | during muscle contraction. |
| | People with this result can put on an extraordinary amount of muscle |
| | mass with the right exercise and nutritional program. Typical people in |
| | this category would be linebackers, power lifters and bodybuilders. |
| | Workout programs would include training programs that would be |
| | targeting athletic performance over fat loss or conditioning. |
| | If you are among the 10% population and want to be really lean and thin |
| | you would want to focus on high intensity interval training vs. 8-10 reps |
| | of heavy compound movements. |
| | Consider working with a certified personal trainer and consult your |
| | physician before undertaking any new exercise programs. |

Metabolism
Fat Processing
Ability

| | |
|---|---|
| Measurement outcome: | This result is the most common and found in 45% of the population. |
| CC | This gene codes a protein that binds to the fatty acids in your diet. |
| | Studies have shown that this CC variant is associated with normal fat |
| | metabolism and a lower risk of obesity. |
| Measurement outcome: | People like you have an uncommon result found in 15% of the |
| TT | population. Studies have shown that people with TT may be the most |
| | sensitive to saturated fatty acids and therefore have a higher risk of |
| | obesity. |
| Measurement outcome: | This result is almost as common as CC and found in 40% of the |
| CT | population. People like you tend to have moderate to high sensitivity to |
| | saturated fats with less ability to metabolize fat consumed. Studies have |
| | shown that the risk of obesity is higher for people with CT than people |
| | with CC. |

Feeling Full

| | |
|---|---|
| Measurement outcome: | You have a genetic variant common to 40% of the population. This gene |
| TT | is known to regulate expression of genes involved in appetite. Studies |
| | have shown that people with TT have the lowest chance compared to |
| | people with AA or AT of being overweight and generally will have a |
| | normal BMI. Strive to eat a healthy, nutrient dense and balanced diet to |
| | meet your needs and goals. |
| Measurement outcome: | This is a rare genetic variant that is found in approximately 20% of the |
| AA | population. This gene's function is not completely understood but one |
| | role it has is it regulates genes that control appetite. Studies with people |
| | of differing weights and body mass index (BMI) have shown that people |
| | with this variant usually have a greater appetite than those with AT or |
| | TT and therefore may have a tendency to gain weight. It is important |
| | build strategies around meals and snacks to be mindful and intuitive to |
| | your hunger and satiety cues. It will be important to try to eat meals at a |
| | slower pace, chewing completely and putting down your fork between |
| | bites to allow time for satiation to be cued. If you struggle with weight |
| | and satiation, you may need a Registered Dietitian Nutritionist to make a |
| | meal plan as a guidance for meal size and frequency based on your |
| | personal needs. |
| Measurement outcome: | This variant is found in 40% of the population. Studies have shown that |
| AT | people with this variant usually have greater appetites than people with |
| | TT leading to higher weight and BMI. It is important build strategies |
| | around meals and snacks to be mindful and intuitive to your hunger and |
| | satiety cues. It will be important to try to eat meals at a slower pace, |
| | chewing completely and putting down your fork between bites to allow |
| | time for satiation to be cued. If you struggle with weight and satiation, |
| | you may need a Registered Dietitian Nutritionist to make a meal plan as |
| | a guidance for meal size and frequency based on your personal needs. |

Monounsaturated
Fat

| | |
|---|---|
| Measurement outcome: | You have a result of this gene that is even more rare than people who |
| GG | have CG. Studies have shown that this gene is associated with greater |
| | weight gain in response to a high fat diet. It is important to be mindful of |
| | the amount and type of fat you consume in your diet, to help prevent |
| | weight gain. Choose healthy fats in moderate amounts from nuts, seeds, |
| | oils, avocado and fish rich in omega-3 fatty acids. Limit or avoid fried |
| | foods and high-fat meat and dairy products. Consider working with a |
| | Registered Dietitian Nutritionist that can help you develop a meal plan |
| | that includes the right amount of fat to help aid in attaining or |
| | maintaining a healthy weight. |
| Measurement outcome: | You have a gene result very common to over 90% of the population. |
| CC | This gene has a function in controlling the storage of fat in your body |
| | and your likelihood to gain weight. People like you have normal fat |
| | storage and therefore normal metabolism. It is recommended you follow |
| | a healthy, nutrient dense and balanced diet to maintain health and reduce |
| | risk of chronic disease. |
| Measurement outcome: | This is a rare result of this gene. People with this result may metabolize |
| CG | fat from the diet more poorly than people with CC. Studies have shown |
| | that people with this result may gain weight more easily. It is important |
| | to be mindful of the amount and type of fat you consume in your diet, to |
| | help prevent weight gain. Choose healthy fats in moderate amounts from |
| | nuts, seeds, oils, avocado and fish rich in omega-3 fatty acids. Limit or |
| | avoid fried foods and high-fat meat and dairy products. Consider |
| | working with a Registered Dietitian Nutritionist that can help you |
| | develop a meal plan that includes the right amount of fat to help aid in |
| | attaining or maintaining a healthy weight. |

Joint Health
Joint Health and
Injury

| | |
|---|---|
| Measurement outcome: | This gene codes for a protein that is a growth factor and keeps your |
| AG | bones and joints strong. You have a result similar to AA that is found in |
| | about 40% of the population. Studies have shown that people with this |
| | result may be more prone to joint injuries. |
| | Consider a workout program tailored to improving your joint health. |
| | Exercises such as one leg balances, rotator cuff exercises, double leg |
| | bridges, squats, hamstring curls and supermans can be beneficial. |
| | Consider a training program that balances low and high impact activities |
| | to strengthen muscles will help protect from injury. Also avoid any |
| | behind the neck press activities. Consider swimming as an alternative to |
| | running sports. |
| | [NOTE]-If you are gifted in genes within the endurance category be |
| | extra careful not to push too far with this result. Its advised to also do |
| | more pre-conditioning and dynamic stretching work than normal with |
| | this result. People with this result may also want to consider shortening a |
| | normal workout before muscles are fatigued if you are gifted in any |
| | endurance categories to help prevent injuries. |
| | Nutritional supplements can also be beneficial for joint health. Some |
| | examples of supplements shown in medical studies to be beneficial are |
| | chondroitin sulfate, glucosamine, and hyaluronic acid. |
| | Consider working with a certified personal trainer and consult your |
| | physician before undertaking any new exercise programs. |
| Measurement outcome: | This gene codes for a protein that is a growth factor and keeps your |
| GG | bones and joints strong. You have a result that is found in only 20% of |
| | the population. Studies have shown that people with this result are more |
| | likely to have stronger joints than people with AA or AG types. |
| | Even though with this result joint injury risk may be lower its still |
| | advisable to be especially careful if you are gifted in any one or multiple |
| | endurance genes. Nutritional supplements can also be beneficial for |
| | joint health. Some examples of supplements shown in medical studies to |
| | be beneficial are chondroitin sulfate, glucosamine, and hyaluronic acid. |
| | Consider working with a certified personal trainer and consult your |
| | physician before undertaking any new exercise programs. |
| Measurement outcome: | You have a result similar to AG that is found in about 40% of the |
| AA | population. This gene codes for a protein that is a growth factor and |
| | keeps your bones and joints strong. Studies have shown that people with |
| | this result may have an increased risk of joint injuries. |
| | Consider a workout program tailored to improving your joint health. |
| | Exercises such as one leg balances, rotator cuff exercises, double leg |
| | bridges, squats, hamstring curls and supermans can be beneficial. |
| | [NOTE]-If you are gifted in genes within the endurance category be |
| | extra careful not to push too far with this result. Its advised to also do |
| | more pre-conditioning and dynamic stretching work than normal with |
| | this result. People with this result may also want to consider shortening a |
| | normal workout before muscles are fatigued if you are gifted in any |
| | endurance categories to help prevent injuries. |
| | Consider working with a certified personal trainer and consult your |
| | physician before undertaking any new exercise programs. |

TABLE 4
FUEL ™ Example Descriptions
FUEL ™
Food Breakdown
Monounsaturated
Fat

| | |
|---|---|
| Measurement outcome: | You have a result of this gene that is even more rare than people who |
| GG | have CG. Studies have shown that this gene is associated with greater |
| | weight gain in response to a high fat diet. It is important to be mindful |
| | of the amount and type of fat you consume in your diet, to help prevent |
| | weight gain. Choose healthy fats in moderate amounts from nuts, seeds, |
| | oils, avocado and fish rich in omega-3 fatty acids. Limit or avoid fried |
| | foods and high-fat meat and dairy products. Consider working with a |
| | Registered Dietitian Nutritionist that can help you develop a meal plan |
| | that includes the right amount of fat to help aid in attaining or |
| | maintaining a healthy weight. |
| Measurement outcome: | You have a gene result very common to over 90% of the population. |
| CC | This gene has a function in controlling the storage of fat in your body |
| | and your likelihood to gain weight. People like you have normal fat |
| | storage and therefore normal metabolism. It is recommended you |
| | follow a healthy, nutrient dense and balanced diet to maintain health |
| | and reduce risk of chronic disease. |
| Measurement outcome: | This is a rare result of this gene. People with this result may metabolize |
| CG | fat from the diet more poorly than people with CC. Studies have shown |
| | that people with this result may gain weight more easily. It is important |
| | to be mindful of the amount and type of fat you consume in your diet, |
| | to help prevent weight gain. Choose healthy fats in moderate amounts |
| | from nuts, seeds, oils, avocado and fish rich in omega-3 fatty acids. |
| | Limit or avoid fried foods and high-fat meat and dairy products. |
| | Consider working with a Registered Dietitian Nutritionist that can help |
| | you develop a meal plan that includes the right amount of fat to help aid |
| | in attaining or maintaining a healthy weight. |

Vitamins
Folate Levels

| | |
|---|---|
| Measurement outcome: | 35% of the population are GA. You may have reduced levels of folate |
| GA | (65% of normal) and therefore should make sure you eat enough folate |
| | rich foods such as lentils, beans, asparagus, spinach, turnip greens, |
| | broccoli, and beets and may consider an additional folate supplement, |
| | as needed and discussed with your doctor. |
| Measurement outcome: | You have a variant found in 55% of the population and is associated |
| GG | with normal levels of folate. You should continue to eat a well balanced |
| | diet including foods rich in folate. You may consider discussing with |
| | your Doctor additional folate supplementation if you are a female of |
| | child-bearing age. |
| Measurement outcome: | This is the rare result found in 10% of the population. You have much |
| AA | reduced levels of the enzyme (30% of normal). This will give you lower |
| | levels of folate, Vitamin B12, and higher levels of homocysteine. |
| | Therefore you should actively seek food such as lentils, beans, |
| | asparagus, spinach, turnip greens, broccoli, and beets that are rich in |
| | folate and may consider an additional folate supplement, as needed and |
| | discussed with your doctor. |

Vitamin B12

| | |
|---|---|
| Measurement outcome: | You fall into the majority of 50% of the population that has this result. |
| GG | With this result you are likely to have low levels of Vitamin B12. Low |
| | levels of Vitamin B12 are due to poor absorption in the intestine. The |
| | GG result is also associated with lower levels of certain gut bacteria. It |
| | is recommended to discuss your Vitamin B12 levels with your |
| | physician and discuss possible supplementation of B12. |
| Measurement outcome: | This result is found in 35% of the population and is associated with |
| AG | intermediate levels of Vitamin B12 levels in your blood. It is |
| | recommended to discuss your Vitamin B12 levels with your physician |
| | and discuss possible supplementation of B12. |
| Measurement outcome: | This is a rare result, about 15% of the population has AA. Research has |
| AA | shown that this result has naturally high levels of Vitamin B12 so you |
| | are not likely to suffer from vitamin B12 deficiency. You will likely get |
| | enough Vitamin B12 from the food you eat. You are also more likely to |
| | be resistant to certain infections. |

Vitamin B6

| | |
|---|---|
| Measurement outcome: | 40% of the population have this genotype that is associated with lower |
| CC | levels of Vitamin B6. This is likely due to higher clearance of Vitamin |
| | B6 from your body. Severe shortage of Vitamin B6 can lead to serious |
| | health issues. It's important to eat foods that have high sources of |
| | Vitamin B6 such as fish, meat, whole grains, nuts and fruit and discuss |
| | with your Doctor if supplementation is needed. |
| Measurement outcome: | 40% of the population have this genotype and you likely have |
| CT | intermediate levels of Vitamin B6. It's important to eat enough foods |
| | that contain Vitamin B6, such as fish, meat, whole grains, nuts and |
| | fruit. |
| Measurement outcome: | This is the rare genotype found in 20% of the population that is |
| TT | associated with normally higher levels of Vitamin B6. Unlike CC and |
| | CT, you have a naturally higher level of Vitamin B6. You are less prone |
| | to a Vitamin B6 deficiency. |

Vitamin E

| | |
|---|---|
| Measurement outcome: | This is a rare result but not as rare as GG, it's found in 5% of the |
| GA | population. You may naturally have low levels of Vitamin E and |
| | therefore need to make sure you eat Vitamin E containing foods. |
| Measurement outcome: | You are common as part of 93% of the population and may have higher |
| AA | levels of Vitamin E which is protective. However, you need to avoid |
| | eating too many foods rich in Vitamin E since too much Vitamin E is |
| | harmful. |
| Measurement outcome: | This is the rare result shared with 2% of the population. Your Vitamin |
| GG | E levels are likely to be lower and you may want to make sure you eat |
| | foods containing Vitamin E. |

Vitamin C

| | |
|---|---|
| Measurement outcome: | This is a very rare type. Studies have shown that you may have lower |
| TT | levels of Vitamin C in your blood suggesting that you should eat many |
| | foods high in Vitamin C. Lower levels of Vitamin C in the body may |
| | cause adverse health risks, consult with your doctor to find out your |
| | Vitamin C levels and adjust dietary intake as needed. |
| Measurement outcome: | This is rare type found in 9% of the population and may be associated |
| CT | with intermediate levels of Vitamin C in your blood. Lower levels of |
| | Vitamin C in the body may cause adverse health risks, consult with |
| | your doctor to find out your Vitamin C levels and adjust dietary intake |
| | as needed. |
| Measurement outcome: | This is the common form of this gene with about 90% of the population |
| CC | having CC. You are likely to have well balanced levels of Vitamin C. |
| | Enjoy Vitamin C rich foods from fruits and vegetables in your meals |
| | and snacks. |

Beta Carotene,
Vitamin A

| | |
|---|---|
| Measurement outcome: | You are among the 39% of the population with a reduced conversion of |
| AT | beta carotene to retinol. You may benefit from eating more foods rich in |
| | Vitamin A and may discuss with your doctor the use of a Vitamin A |
| | supplement (in the form of beta carotene or mixed carotenoids). Food |
| | sources of Vitamin A include carrots, kale, spinach, cantaloupe, |
| | apricots, mango, whole eggs, milk and liver. |
| Measurement outcome: | 56% of the population have a normal conversion rate and therefore if |
| AA | you are eating a balanced and varied diet rich in Vitamin A, your levels |
| | should be adequate. Food sources of Vitamin A include carrots, kale, |
| | spinach, cantaloupe, apricots, mango, whole eggs, milk and liver. |
| Measurement outcome: | You are very rare with 5% of the population and you are similar to |
| TT | “AT” and have a reduced conversion of beta carotene to retinol. You |
| | may benefit from eating more foods rich in Vitamin A and may discuss |
| | with your doctor the use of a Vitamin A supplement (in the form of beta |
| | carotene or mixed carotenoids). Food sources of Vitamin A, include |
| | carrots, kale, spinach, cantaloupe, apricots, mango, whole eggs, milk |
| | and liver. |

Vitamin D

| | |
|---|---|
| Measurement outcome: | You are similar to 60% of the population and are likely to have normal |
| TT | Vitamin D levels. The TT type is not known to be associated with |
| | Vitamin D deficiency. Follow guidelines for normal sun exposure for |
| | health and to maintain a normal Vitamin D level. It is still advised to |
| | speak with your Doctor about receiving the 25 (OH) D blood test to |
| | learn your Vitamin D levels. |
| Measurement outcome: | 35% of the population has TG and you may have levels of vitamin D |
| TG | between those with TT and GG. Follow guidelines for short exposure |
| | to the sun to help increase your Vitamin D levels, and increase foods |
| | that are good sources of Vitamin D such as salmon, tuna and eggs. Talk |
| | to your Doctor about receiving the 25 (OH) D blood test to learn your |
| | Vitamin D levels and possibly supplement with a Vitamin D3 |
| | supplement at a level recommended from your Doctor. |
| Measurement outcome: | You are among 5% of the population that may have reduced vitamin D |
| GG | levels. Follow guidelines for short exposure to the sun to help increase |
| | your Vitamin D levels, and increase foods that are good sources of |
| | Vitamin D such as salmon, tuna and eggs. Talk to your Doctor about |
| | receiving the 25 (OH) D blood test to learn your Vitamin D levels and |
| | possibly supplement with a Vitamin D3 supplement at a level |
| | recommended from your Doctor. |

### Computer System and Network Environment

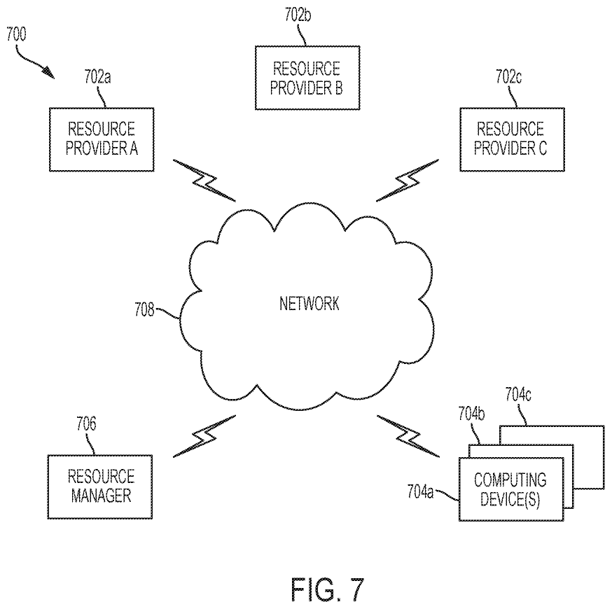

FIG. 7 shows an illustrative network environment 700 for use in the methods and systems described herein. In brief overview, referring now to FIG. 7, a block diagram of an exemplary cloud computing environment 700 is shown and described. The cloud computing environment 700 may include one or more resource providers 702a, 702b, 702c (collectively, 702). Each resource provider 702 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 702 may be connected to any other resource provider 702 in the cloud computing environment 700. In some implementations, the resource providers 702 may be connected over a computer network 708. Each resource provider 702 may be connected to one or more computing device 704a, 704b, 704c (collectively, 704), over the computer network 708.

The cloud computing environment 700 may include a resource manager 706. The resource manager 706 may be connected to the resource providers 702 and the computing devices 704 over the computer network 708. In some implementations, the resource manager 706 may facilitate the provision of computing resources by one or more resource providers 702 to one or more computing devices 704. The resource manager 706 may receive a request for a computing resource from a particular computing device 704. The resource manager 706 may identify one or more resource providers 702 capable of providing the computing resource requested by the computing device 704. The resource manager 706 may select a resource provider 702 to provide the computing resource. The resource manager 706 may facilitate a connection between the resource provider 702 and a particular computing device 704. In some implementations, the resource manager 706 may establish a connection between a particular resource provider 702 and a particular computing device 704. In some implementations, the resource manager 706 may redirect a particular computing device 704 to a particular resource provider 702 with the requested computing resource.

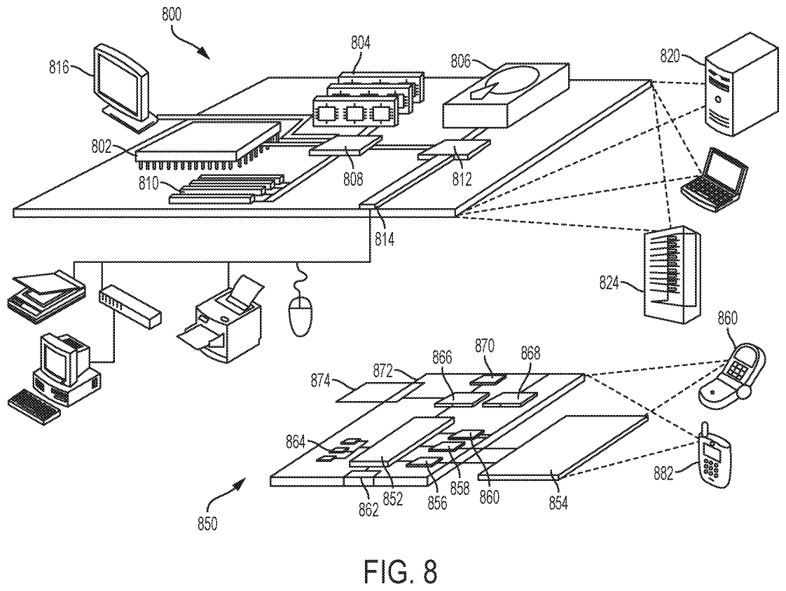

FIG. 8 shows an example of a computing device 800 and a mobile computing device 850 that can be used in the methods and systems described in this disclosure. The computing device 800 is intended to represent various forms of digital computers, such as laptops, desktops, workstations, personal digital assistants, servers, blade servers, mainframes, and other appropriate computers. The mobile computing device 850 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 800 includes a processor 802, a memory 804, a storage device 806, a high-speed interface 808 connecting to the memory 804 and multiple high-speed expansion ports 810, and a low-speed interface 812 connecting to a low-speed expansion port 814 and the storage device 806. Each of the processor 802, the memory 804, the storage device 806, the high-speed interface 808, the high-speed expansion ports 810, and the low-speed interface 812, are interconnected using various busses, and may be mounted on a common motherboard or in other manners as appropriate. The processor 802 can process instructions for execution within the computing device 800, including instructions stored in the memory 804 or on the storage device 806 to display graphical information for a GUI on an external input/output device, such as a display 816 coupled to the high-speed interface 808. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system). Thus, as the term is used herein, where a plurality of functions are described as being performed by “a processor”, this encompasses embodiments wherein the plurality of functions are performed by any number of processors (one or more) of any number of computing devices (one or more). Furthermore, where a function is described as being performed by “a processor”, this encompasses embodiments wherein the function is performed by any number of processors (one or more) of any number of computing devices (one or more) (e.g., in a distributed computing system).

The memory 804 stores information within the computing device 800. In some implementations, the memory 804 is a volatile memory unit or units. In some implementations, the memory 804 is a non-volatile memory unit or units. The memory 804 may also be another form of computer-readable medium, such as a magnetic or optical disk.

The storage device 806 is capable of providing mass storage for the computing device 800. In some implementations, the storage device 806 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 802), perform one or more methods, such as those described above. The instructions can also be stored by one or more storage devices such as computer- or machine-readable mediums (for example, the memory 804, the storage device 806, or memory on the processor 802).

The high-speed interface 808 manages bandwidth-intensive operations for the computing device 800, while the low-speed interface 812 manages lower bandwidth-intensive operations. Such allocation of functions is an example only. In some implementations, the high-speed interface 808 is coupled to the memory 804, the display 816 (e.g., through a graphics processor or accelerator), and to the high-speed expansion ports 810, which may accept various expansion cards (not shown). In the implementation, the low-speed interface 812 is coupled to the storage device 806 and the low-speed expansion port 814. The low-speed expansion port 814, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 800 may be implemented in a number of different forms, as shown in the figure. For example, it may be implemented as a standard server 820, or multiple times in a group of such servers. In addition, it may be implemented in a personal computer such as a laptop computer 822. It may also be implemented as part of a rack server system 824. Alternatively, components from the computing device 800 may be combined with other components in a mobile device (not shown), such as a mobile computing device 850. Each of such devices may contain one or more of the computing device 800 and the mobile computing device 850, and an entire system may be made up of multiple computing devices communicating with each other.

The mobile computing device 850 includes a processor 852, a memory 864, an input/output device such as a display 854, a communication interface 866, and a transceiver 868, among other components. The mobile computing device 850 may also be provided with a storage device, such as a micro-drive or other device, to provide additional storage. Each of the processor 852, the memory 864, the display 854, the communication interface 866, and the transceiver 868, are interconnected using various buses, and several of the components may be mounted on a common motherboard or in other manners as appropriate.

The processor 852 can execute instructions within the mobile computing device 850, including instructions stored in the memory 864. The processor 852 may be implemented as a chipset of chips that include separate and multiple analog and digital processors. The processor 852 may provide, for example, for coordination of the other components of the mobile computing device 850, such as control of user interfaces, applications run by the mobile computing device 850, and wireless communication by the mobile computing device 850.

The processor 852 may communicate with a user through a control interface 858 and a display interface 856 coupled to the display 854. The display 854 may be, for example, a TFT (Thin-Film-Transistor Liquid Crystal Display) display or an OLED (Organic Light Emitting Diode) display, or other appropriate display technology. The display interface 856 may comprise appropriate circuitry for driving the display 854 to present graphical and other information to a user. The control interface 858 may receive commands from a user and convert them for submission to the processor 852. In addition, an external interface 862 may provide communication with the processor 852, so as to enable near area communication of the mobile computing device 850 with other devices. The external interface 862 may provide, for example, for wired communication in some implementations, or for wireless communication in other implementations, and multiple interfaces may also be used.

The memory 864 stores information within the mobile computing device 850. The memory 864 can be implemented as one or more of a computer-readable medium or media, a volatile memory unit or units, or a non-volatile memory unit or units. An expansion memory 874 may also be provided and connected to the mobile computing device 850 through an expansion interface 872, which may include, for example, a SIMM (Single In Line Memory Module) card interface. The expansion memory 874 may provide extra storage space for the mobile computing device 850, or may also store applications or other information for the mobile computing device 850. Specifically, the expansion memory 874 may include instructions to carry out or supplement the processes described above, and may include secure information also. Thus, for example, the expansion memory 874 may be provided as a security module for the mobile computing device 850, and may be programmed with instructions that permit secure use of the mobile computing device 850. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier and, when executed by one or more processing devices (for example, processor 852), perform one or more methods, such as those described above. The instructions can also be stored by one or more storage devices, such as one or more computer- or machine-readable mediums (for example, the memory 864, the expansion memory 874, or memory on the processor 852). In some implementations, the instructions can be received in a propagated signal, for example, over the transceiver 868 or the external interface 862.

The mobile computing device 850 may communicate wirelessly through the communication interface 866, which may include digital signal processing circuitry where necessary. The communication interface 866 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 868 using a radio-frequency. In addition, short-range communication may occur, such as using a Bluetooth®, Wi-Fi™, or other such transceiver (not shown). In addition, a GPS (Global Positioning System) receiver module 870 may provide additional navigation- and location-related wireless data to the mobile computing device 850, which may be used as appropriate by applications running on the mobile computing device 850.

The mobile computing device 850 may also communicate audibly using an audio codec 860, which may receive spoken information from a user and convert it to usable digital information. The audio codec 860 may likewise generate audible sound for a user, such as through a speaker, e.g., in a handset of the mobile computing device 850. Such sound may include sound from voice telephone calls, may include recorded sound (e.g., voice messages, music files, etc.) and may also include sound generated by applications operating on the mobile computing device 850.

The mobile computing device 850 may be implemented in a number of different forms, as shown in the figure. For example, it may be implemented as a cellular telephone 880. It may also be implemented as part of a smart-phone 882, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

While the invention has been particularly shown and described with reference to specific preferred embodiments, it should be understood by those skilled in the art that various changes in form and detail may be made therein without departing from the spirit and scope of the invention as defined by the appended claims.

What is claimed is:

1. A method for providing individualized purchase recommendations based on a genetic profile assessment of an individual the method comprising:
reading, by a processor of a computing device, genotyping data corresponding to a biological sample of a user, wherein the genotyping data comprises one or more genotyping measurements of one or more SNPs, each SNP having a genetic position and being associated with one or more genes;
accessing, by the processor, a purchase recommendation database, the purchase recommendation database comprising one or more purchase recommendation objects, each purchase recommendation objects comprising a data structure, each data structure corresponding to a product and comprising one or more categories hierarchically inferior to the product, each category having one or more related gene objects hierarchically inferior to the category, each gene object having one or more SNP objects hierarchically inferior to the gene object, each SNP object having one or more variants hierarchically inferior to the gene object, each SNP object having a health-related phenotype, and each variant having a qualifier hierarchically inferior to the variant;
determining, by the processor, one or more recommended products by matching one or more measured SNPs from the one or more genotyping measurements to one or more variants within the data structures of the one or more products in the purchase recommendation database; and
rendering, by the processor, one or more icons and/or alphanumeric strings corresponding to the one or more recommended products.

2. The method of claim 1, further comprising graphically rendering, by the processor, an assessment GUI view comprising a graphical representation of the one or more SNP with which the one or more recommended products purchase is associated; and
wherein rendering the one or more icons and/or alphanumeric strings corresponding to the one or more recommended products within the assessment GUI view in a manner that visually associates the one or more icons and/or alphanumeric strings with the graphical representation of the results of the genotyping measurement of the one or more SNP.

3. The method of claim 1, wherein the one or more recommended products comprise one or more supplements.

4. The method of claim 1, wherein: matching the one or more measured SNPs from the one or more genotyping measurements to one or more variants comprises, for each matching pair comprising a variant matched to a measured SNP: determining that the variant comprises a higher susceptibility to a negative health-related trait, wherein the one or more recommended products are associated with benefits for users with the negative health-related trait.

5. A system for providing individualized purchase recommendations based on a genetic profile assessment of an individual, the system comprising:
a processor; and
a memory having instructions stored thereon, wherein the instructions, when executed by the processor, cause the processor to:
reading genotyping data corresponding to a biological sample of a user, wherein the genotyping data comprises one or more genotyping measurements of one or more SNPs, each SNP having a genetic position and being associated with one or more genes;
accessing a purchase recommendation database, the purchase recommendation database comprising one or more purchase recommendation objects, each purchase recommendation objects comprising a data structure, each data structure corresponding to a product and comprising one or more categories hierarchically inferior to the product, each category having one or more related gene objects hierarchically inferior to the category, each gene object having one or more SNP objects hierarchically inferior to the gene object, each SNP object having one or more variants hierarchically inferior to the gene object, each SNP object having a health-related phenotype, and each variant having a qualifier hierarchically inferior to the variant;
determining one or more recommended products by matching one or more measured SNPs from the one or more genotyping measurements to one or more variants within the data structures of the one or more products in the purchase recommendation database; and
rendering one or more icons and/or alphanumeric strings corresponding to the one or more recommended products.
